SEQUENCE LISTING (2 sequences)

SEQ ID NO: 1 - protein, 613 aa, Homo sapiens
MEIALVPLENGGAMTVRGGDEARAGCGQATGGELQCPPTAGLSDGPKEPAPKGRGAQRDADSGVRPLPPLPDPGVRPLPPLPEELPRPRRPPPEDEEEEGDPGLGTVEDQALGTASLHHQRVHINISGLRFETQLGTLAQFPNTLLGDPAKRLRYFDPLRNEYFFDRNRPSFDGILYYYQSGGRLRRPVNVSLDVFADEIRFYQLGDEAMERFREDEGFIKEEEKPLPRNEFQRQVWLIFEYPESSGSARAIAIVSVLVILISIITFCLETLPEFRDERELLRHPPAPHQPPAPAPGANGSGVMAPPSGPTVAPLLPRTLADPFFIVETTCVIWFTFELLVRFFACPSKAGFSRNIMNIIDVVAIFPYFITLGTELAEQQPGGGGGGQNGQQAMSLAILRVIRLVRVFRIFKLSRHSKGLQILGKTLQASMRELGLLIFFLFIGVILFSSAVYFAEADNQGTHFSSIPDAFWWAVVTMTTVGYGDMRPITVGGKIVGSLCAIAGVLTIALPVPVIVSNFNYFYHRETDHEEPAVLKEEQGTQSQGPGLDRGVQRKVSGSRGSFCKAGGTLENADSARRGSCPLEKCNVKAKSNVDLRRSLYALCLDTSRETDL

SEQ ID NO: 2 - DNA, 1842 bp, Homo sapiens
atggagatcgccctggtgcccctggagaacggcggtgccatgaccgtcagaggaggcgatgaggcccgggcaggctgcggccaggccacagggggagagctccagtgtcccccgacggctgggctcagcgatgggcccaaggagccggcgccaaaggggcgcggcgcgcagagagacgcggactcgggagtgcggcccttgcctccgctgccggacccgggagtgcggcccttgcctccgctgccagaggagctgccacggcctcgacggccgcctcccgaggacgaggaggaagaaggcgatcccggcctgggcacggtggaggaccaggctctgggcacggcgtccctgcaccaccagcgcgtccacatcaacatctccgggctgcgctttgagacgcagctgggcaccctggcgcagttccccaacacactcctgggggaccccgccaagcgcctgcgctacttcgaccccctgaggaacgagtacttcttcgaccgcaaccggcccagcttcgacggtatcctctactactaccagtccgggggccgcctgcggaggccggtcaacgtctccctggacgtgttcgcggacgagatacgcttctaccagctgggggacgaggccatggagcgcttccgcgaggatgagggcttcattaaagaagaggagaagcccctgccccgcaacgagttccagcgccaggtgtggcttatcttcgagtatccggagagctctgggtccgcgcgggccatcgccatcgtctcggtcttggttatcctcatctccatcatcaccttctgcttggagaccctgcctgagttcagggatgaacgtgagctgctccgccaccctccggcgccccaccagcctcccgcgcccgcccctggggccaacggcagcggggtcatggccccgccctctggccctacggtggcaccgctcctgcccaggaccctggccgaccccttcttcatcgtggagaccacgtgcgtcatctggttcaccttcgagctgctcgtgcgcttcttcgcctgccccagcaaggcagggttctcccggaacatcatgaacatcatcgatgtggtggccatcttcccctacttcatcaccctgggcaccgaactggcagagcagcagccagggggtggaggaggcggccagaatgggcagcaggccatgtccctggccatcctccgagtcatccgcctggtccgggtgttccgcatcttcaagctctcccgccactccaaggggctgcagatcctgggcaagaccttgcaggcctccatgagggagctggggctgctcatcttcttcctcttcatcggggtcatcctcttctccagtgccgtctacttcgcagaggctgacaaccagggaacccatttctctagcatccctgacgccttctggtgggcagtggtcaccatgaccactgtgggctacggggacatgaggcccatcactgttgggggcaagatcgtgggctcgctgtgtgccatcgccggggtcctcaccattgccctgcctgtgcccgtcatcgtctccaacttcaactacttctaccaccgggaaacggatcacgaggagccggcagtccttaaggaagagcagggcactcagagccaggggccggggctggacagaggagtccagcggaaggtcagcgggagcaggggatccttctgcaaggctggggggaccctggagaatgcagacagtgcccgaaggggcagctgccccctagagaagtgtaacgtcaaggccaagagcaacgtggacttgcggaggtccctttatgccctctgcctggacaccagccgggaaacagatttgtga

(19) United States
(12) Patent Application Publication
(10) Pub. No.: US 2009/0053721 A1
(43) Pub. Date: Feb. 26, 2009

(54) VOLTAGE-GATED ION CHANNEL MUTANTS FOR USE IN IDENTIFYING ION CHANNEL MODULATING COMPOUNDS

(75) Inventor: David Fedida, Vancouver (CA)

Correspondence Address: SEED INTELLECTUAL PROPERTY LAW GROUP PLLC, 701 FIFTH AVE, SUITE 5400, SEATTLE, WA, 98104, US

(73) Assignee: CARDIOME PHARMA CORP., Vancouver (CA)

(21) Appl. No.: 12/188,899

(22) Filed: Aug. 8, 2008

Related U.S. Application Data
(60) Provisional application No. 60/955,290, filed on Aug. 10, 2007.

(51) Int. Cl. C12Q 1/68; G01N 33/68; C07D 211/34; C07D 207/12; C12Q 1/02

(52) U.S. Cl. 435/6; 436/86; 436/89; 435/29; 546/233; 548/556

(57) ABSTRACT

The present invention provides novel methods and compositions for identifying ion channel modulating compounds, including atrial-selective antiarrhythmic agents and ion channel modulating compounds that preferentially modulate K⁺ channels. The ion channel modulating compounds identified according to these methods are associated with a reduced incidence of ventricular proarrhythmia.

### CROSS-REFERENCE TO RELATED APPLICATION

This application claims the benefit under 35 U.S.C. § 119(e) of U.S. Provisional Patent Application No. 60/955,290 filed Aug. 10, 2007, where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480102_{—}459_SEQUENCE_LISTING.txt. The text file is 8 KB, was created on Aug. 8, 2008, and is being submitted electronically via EFS-Web, concurrent with the filing of the specification.

### BACKGROUND

1. Technical Field

This invention is related to mutated voltage-gated ion channels, cells comprising the same, and their use in identifying ion channel modulating compounds.

2. Description of the Related Art

Atrial fibrillation (AF), the most common sustained cardiac arrhythmia, is associated with ˜15% of all strokes (Kannel et al, 1998; Go et al., 2001; Rockson and Albers, 2004) and occurs in about 30% of patients after cardiac surgery (Leung et al., 2004). Currently, the development or exacerbation of AF often prompts emergency department presentations. Although there are a variety of management strategies for acute atrial fibrillation and the outcomes are generally good, catastrophic events can occur as a result of atrial fibrillation, including congestive heart failure, thrombo-embolic phenomena (particularly strokes), ventricular proarrthythmias, and serious adverse effects associated with treatment.

The underlying causes of cardiac arrhythmias are abnormal impulse formation or impulse propagation resulting from defective function of cardiac ion channels and exchangers, with the most common mechanism of AF being complex atrial re-entry. K⁺ channel inhibiting drugs prevent or terminate re-entry by increasing the affected cells' refractory period. Traditional K⁺ channel blocking drugs target I_{Kr}, a channel crucial for repolarization throughout the heart, and therefore affecting ion channels in both atrial and ventricular tissue. Thus, in certain predisposed patients, I_{Kr} blockers stabilize ventricular repolarization, leading to early after depolarization (EAD) related arrhythmias, such as Torsades de Pointes. Accordingly, there is a need for atrial selective antiarrhythmic drugs.

The discovery of an ultra-rapid delayed rectifier potassium current (I_{Kur}) functionally present only in the atrium provided a new target for atrial selective antiarrhythmic drugs. I_{Kur} blockers should delay atrial repolarization and the refractory period without affecting ventricular tissue and, therefore, be free of risk of ventricular pro-arrhythmia. The I_{Kur} current results from expression of the KCNA5 gene and Kv1.5 protein in humans (Fedida et al., 1993; Feng et al., 1997).

Vernakalant (RSD1235) is a mixed frequency-dependent Na⁺ and atria-preferred K⁺ channel blocker (Roy et al., 2004; Fedida et al., 2005) under development for treatment and prevention of a variety of indications, including the acute conversion of atrial fibrillation to sinus rhythm. In recent phase II and III clinical trials, vernakalant has shown promise as an intravenous antiarrhythmic agent for rapid conversion of AF to sinus rhythm with an overall rate close to 52% within 90 min of infusion, compared with a placebo conversion rate of just 3.8% (Roy et al., 2005; Pratt et al., 2006; Stiell et al., 2006; Fedida, 2007). One of the actions of vernakalant is block of the atrial specific I_{Kur} current (Fedida et al., 2005), and evidence suggests that the safety of vernakalant is in part related to the higher sensitivity of atrial-specific Kv1.5 to block by vernakalant over other channels involved in ventricular repolarization, such as hERG (Fedida et al., 2005) and KCNQ1 (unpublished data).

Several other antiarrhythmic drugs in development, including S0100176 (Decher et al., 2004), AVE0118 (Decher et al., 2006), AZD7009 (Persson et al., 2005), as well as some well-known agents, quinidine (Snyders et al., 1992; Fedida, 1997), and flecamide (Grissmer et al., 1994), and the local anesthetics bupivacaine (Franqueza et al., 1997) and benzocaine (Caballero et al., 2002), have also been shown to block Kv1.5. The potency of these agents is affected by introduction of specific mutations in the S6 domain (V505, T507, I508, L510, V512, V514) of Kv1.5, which lines the inner vestibule of the channel, or mutations in the deep pore (T479 and T480) near the selectivity filter (Caballero et al., 2002; Decher et al., 2004; Decher et al., 2006; Herrera et al., 2005; Yeola et al., 1996; Franqueza et al., 1997). Many of these same residues are also important sites of interaction for the Kvβ inactivation particle (Decher et al., 2005), for TEA (Choi et al., 1993; Lopez et al., 1994), and for 4-AP block (Kirsch and Drewe, 1993). Interestingly, it appears that different antiarrythmic agents may interact in different manners or specificities with ion channel proteins such as Kv1.5, thereby leading to different potencies. For example, flecamide, a drug used for the acute conversion of atrial fibrillation to sinus rhythm (Fuster et al., 2006) also blocks Kv1.5 (Grissmer et al., 1994; Herrera et al., 2005), but with a much lower potency than vernakalant.

Given the significant morbidity associated with AF and drug-induced side-effects, there is clearly a need for new atrial-selective antiarrhythmic drugs. The present invention meets this need by providing new methods of identifying compounds having preferential binding specificities to ion channels, such as Kv1.5, which are indicative of enhanced potency, as well as new compositions useful in practicing such methods.

### BRIEF SUMMARY

The present invention provides compositions and methods useful in identifying an ion channel modulating compound. In one embodiment, the present invention provides a method of identifying an ion channel modulating compound, comprising identifying a compound that blocks a Kv1.5 channel comprising a Kv1.5 polypeptide, wherein: (1) said block is reduced when said Kv1.5 polypeptide comprises a mutation in an amino acid residue corresponding to amino acid residue 480, or a region consisting of amino acid residues 500 to 512 of SEQ ID NO:1; and/or (2) said block is increased when said Kv1.5 polypeptide comprises a mutation in an amino acid residue corresponding to amino acid residue 479 or 501 of SEQ ID NO:1. In one embodiment, the Kv1.5 polypeptide comprises the sequence set forth in SEQ ID NO:1 or is a functional variant thereof.

In particular embodiments, said block is reduced at least 5-fold when said Kv1.5 polypeptide comprises a mutation in an amino acid residue corresponding to amino acid residue 502 of SEQ ID NO:1.

In certain embodiments, said block is reduced when said Kv1.5 polypeptide comprises a mutation in an amino acid residue corresponding to an amino acid residue selected from 480, 500, 502, 505, and 508 of SEQ ID NO:1.

In further embodiments, said block is not significantly reduced when said Kv1.5 polypeptide comprises a mutation in a region corresponding to amino acid residues 509-531 of SEQ ID NO:1. In particular embodiments, said Kv1.5 polypeptide comprises a mutation in an amino acid residue corresponding to an amino acid residue selected from 510, 512, and 516 of SEQ ID NO:1.

In further embodiments, said ion channel modulating compound is an atrial-selective antiarrhythmic agent. In related embodiments, said ion channel modulating compound acts preferentially on K⁺ channels as compared to Na⁺ channels. In one embodiment, the said ion channel modulating compound acts preferentially on the I_{Kur} channel as compared to other K+ channels.

In various embodiments, said block is determined in cells expressing an exogenous wild type or mutant Kv1.5 polypeptide. In particular embodiments, said cells comprise an exogenous polynucleotide that encodes a wild type or mutant Kv1.5 polypeptide. In one embodiment, said polynucleotide was introduced into said cells by transient transfection.

In certain embodiments, said block is determined by whole cell current recordings.

In yet a further related embodiment, the present invention includes ion channel modulating compounds identified according to a method of the present invention.

### BRIEF DESCRIPTION OF THE SEVERAL VIEWS OF THE DRAWINGS

FIG. 1 includes two graphs depicting tail current cross-over studies that reveal an inner pore site of access for vernakalant. (A) Voltage protocol for recording of Kv1.5 currents from HEK cells. Cells were held at −80 mV and pulsed to +60 mV for 400 ms before repolarizing to −40 mV to record tail currents. Protocol was repeated at 0.1 Hz intervals. Current traces in the lower panel were obtained in control and after 5 min exposure to 10 μM vernakalant. Inset panel shows tail currents at −40 mV in control and vernakalant on an expanded time base to highlight cross-over. In this figure as in all others, numbers adjacent to tracings refer to drug concentrations in μM unless otherwise stated. (B) Diary plot of steady-state Kv1.5 current at the end of 400 ms depolarizations. Pulse frequency was 0.1 Hz. Between 200 and 380 s the cell was rested at −80 mV while vernakalant was washed out of the bath.

FIG. 2 provides graphs demonstrating Kv1.5 currents in cells containing wild type or the indicated alanine scan mutated forms of pore-S6 of Kv1.5 in the presence of 10 μM vernakalant. (A-L) Kv1.5 currents in control and in the presence of 10 μM vernakalant for each of the indicated mutants. Cells were pulsed from −80 to +60 mV for 400 ms at 0.1 Hz. Channel mutation is identified above each pair of current tracings. (M) Bar graph of fraction of Kv1.5 current block by 10 μM vernakalant. Numbers in brackets denote number of cells studied, and are shown+SEM. **, p<0.05 compared with control block.

FIG. 3 provides graphs depicting the dose-response relationships for vernakalant on selected Kv1.5 mutant channels. (A) Selected current tracings from T479A, I502A, and V505A mutant Kv1.5 channels exposed to increasing concentrations of vernakalant. Cells were held at −80 mV and pulsed to +60 mV as indicated in the protocol at top. In this figure as in all others, numbers adjacent to tracings refer to drug concentrations in μM unless otherwise stated. (B) Dose response relationships obtained from data tracings as in panel A were normalized and plotted vs. log [vernakalant], and fitted to a Hill equation. Data points are shown±SEM. IC₅₀ values may be found in the text and Table I. (C) Bar graph comparing IC₅₀ values for mutant Kv1.5 channels exposed to vernakalant. Numbers in parentheses refer to the number of cells studied, and are shown+SEM. **, p<0.05 compared with control IC₅₀ value.

FIG. 4 provides graphs depicting the dose-response relationships for flecamide on selected Kv1.5 mutant channels. (A) Selected current tracings from Kv1.5 WT, T479A, I502A, I508A, and V505A mutant Kv1.5 channels exposed to increasing concentrations of flecamide. Cells were held at −80 mV and pulsed to +60 mV. (B) Dose response relationships obtained from data tracings as in panel A were normalized and plotted vs. log [flecainide], and fitted to a Hill equation. Data points are shown±SEM. IC₅₀ values may be found in the text and Table I. (C) Bar graph comparing IC₅₀ values for mutant Kv1.5 channels exposed to flecamide. Numbers in parentheses refer to the number of cells studied, and are shown+SEM. **, p<0.05 compared with control IC₅₀ value.

FIG. 5 shows graphs depicting various amino acid substitutions to selected residues in S6, and their block by vernakalant. (A) 3-axis graph of the effect of different amino acid substitutions of the WT residues (abscissa) on the resultant mutant Kv1.5 channel IC₅₀ (ordinate) for block by vernakalant. (B) Correlation graphs comparing vernakalant IC₅₀ (left ordinate in μM) vs. hydrophobicity (right ordinate, Kyte-Doolittle units), and size (left ordinate, Å³) for a range of amino acids substituted at I502 (B), V505 (C), and I508 (D). Spearman correlation coefficients: for I502 for size and hydrophobicity were −0.4, and −1.0, respectively; for V505, r=0.0 and −0.4; for I508, r=−0.4, and −0.6 (−1.0 without tyr).

FIG. 6 shows graphs depicting various amino acid substitutions to selected residues in S6, and their block by flecamide. (A) 3-axis graph of the effect of different amino acid substitutions of the WT residues (abscissa) on the resultant mutant Kv1.5 channel IC₅₀ (ordinate) for block by flecamide. (B-D) Correlation graphs comparing flecamide IC₅₀ (left ordinate in μM) vs. hydrophobicity (right ordinate, Kyte-Doolittle units), and size (left ordinate, Å³) for a range of amino acids substituted at I502 (B), V505 (C), and I508 (D). Spearman correlation coefficients: for I502 for size and hydrophobicity were 0.4, and −0.6, respectively; for V505, r=−0.5, and 0.5; for I508, r=1.00, and 0.5.

FIG. 7 provides graphs depicting the voltage-dependent block of selected pore and S6 mutants by vernakalant. (A) Kv1.5 current tracings in control (above) and during exposure to 10 μM vernakalant (below) at a range of membrane potentials from −80 to +60 mV, as shown in the protocol at top, data from the same cell. (B) Current-voltage relationships obtained from WT and selected mutant Kv1.5 channels as labeled on each graph. Data points were obtained from experiments carried out as illustrated in panel A. Currents measured at the end of clamp pulses (at arrows in A) are plotted±SEM. vs. voltage clamp step potential for different concentrations of vernakalant (in μM). For WT data, n=12, for T479A, n=6; for I502A, n=6; for V505A, n=8; for I508A, n=8. (C) Normalized voltage-dependent block is plotted vs. clamp potential and fitted with a Woodhull equation to calculate the fractional electrical distance of drug binding (δ, see inset legend for values, and Methods and text for explanation).

FIG. 8 provides graphs depicting the voltage-dependent block of selected pore and S6 mutants by flecamide. (A) Kv1.5 Current tracings in control (above) and during exposure to 30 μM flecamide (below) at a range of membrane potentials from −80 to +60 mV, as shown in the protocol at top, data from the same cell. (B) Current-voltage relationships obtained from WT and selected mutant Kv1.5 channels as labeled on each graph. Data points were obtained from experiments carried out as illustrated in panel A. Currents measured at the end of clamp pulses (at arrows in A) are plotted±SEM. vs. voltage clamp step potential for different concentrations of flecamide (in μM). For WT data, n=12, for T479A, n=6; for I502A, n=6; for V505A, n=8; for I508A, n=8. (C) Normalized voltage-dependent block is plotted vs. clamp potential and fitted with a Woodhull equation to calculate the fractional electrical distance of drug binding (δ, see inset legend for values).

FIG. 9 shows a comparison of effects of alanine substitution on block of Kv1.5 by vernakalant, flecamide, and other antiarrythmic agents. Percent changed in IC₅₀ or fractional block were calculated from derived data (vernakalant and flecamide) or from values obtained from readily available published work involving alanine mutations. At residue 501, the residue was an alanine to a valine.

FIG. 10 illustrates a Kv1.5 pore homology model illustrating residues that when mutated altered sensitivity to block by vernakalant, as well as the amino acids VGY which make up part of the selectivity filter. A Kv1.5 homology model was generated for the S5-p-loop-S6 region based on the crystal structure for Kv1.2. (A) The model shows side chains for residues T479, T480, I502, V505 and I508. (B) The side chain of I502 appears to point towards L437 and 441 (shown in space fill representation) in the adjacent subunit. (C) Homology model predicts an H-bond between C500 in S6 and T477 in the pore helix.

FIG. 11 provides an alignment of potential pore interaction regions, including the S5, pore, and S6 regions, from a variety of K channels. Refers to omitted sequence in the proximal pre helix. Identical residues in different channels are identified by (−). The span of the S5, selectivity filter and S6 regions are identified by the solid black bars above the sequence. Residues, I502, V505 and I508 are identified by highlighting in the top row for Kv1.5, and the I502 equivalent residue in the other channels. Underscored residues in KVLQT1 and hERG are discussed below.

### DETAILED DESCRIPTION

The present invention is based upon the identification of residues within Kv1.5 that are involved in vernakalant binding and channel blockage, including residues that distinguish between vernakalant and flecamide binding and channel blockage. As described in detail in the accompanying Examples, the binding site of vernakalant in the deep pore and S6 of Kv1.5 was investigated using both electrophysiological and mutational methods. Flecamide was used as a comparator compound to validate the resulting data against that already present in the literature, and also to extend studies of flecamide block itself. These results were interpreted in the context of the hydrophobicity, size, and potential for cation π interactions of the different substituted residues, and were extended by carrying out homology-modeling of Kv1.5 based on the published crystal structure of Kv1.2 (Long et al., 2005).

The mutation scan identified several highly conserved amino acids, including T479, T480, I502, V505, and I508 of the human Kv1.5 protein (SEQ ID NO:1), as important residues for affecting block by both compounds. In general, mutations in S6 increased the IC₅₀ for block by vernakalant, with I502A causing an extremely local 25-fold decrease in potency. These studies also demonstrated that I502 in the S6 domain is a key residue in the block of Kv1.5 by vernakalant, but less so for flecamide, and that for some of the mutations examined, the direction of changes in IC₅₀ were opposite for vernakalant and flecamide, highlighting differences in the forces that drive drug-channel interactions.

In view of these studies, the present invention provides novel methods of identifying ion channel modulating compounds, including, e.g., compounds useful in the treatment of various arrhythmias, based upon their preferential interactions with specific amino acid residues within Kv1.5. In addition, by examining the effect of various Kv1.5 mutations on the activity of ion channel modulating compounds, the present methods may be used to identify those having preferential properties, such as high potency or high safety due to selective action on Kv1.5 as opposed to other channels. They may also be used to identify compounds having properties similar to other known drugs, such as vernakalant or flecamide. In certain embodiments, atrial-selective ion channel modulating compounds have an increased activity on atrial tissue as compared to ventricular tissue, which may be due to increased binding to or activity against specific ion channels preferentially expressed on atrial tissue as opposed to ventricular tissue.

The present invention also provides mutated Kv1.5 polynucleotides and polypeptides, as well as cells comprising the same, which may be used to screen for ion channel modulating compounds, including atrial-selective drugs and drugs exhibiting binding characteristics more similar to vernakalant than flecamide. In addition, the present invention includes compounds identified according to a method of the present invention.

### Ion Channel Compositions

In light of the discovery of the present invention that different ion channel modulating compounds interact differently with the Kv1.5 channel, the present invention includes a variety of compositions and methods that may be advantageously used to determine how an ion channel modulating compound interacts with the Kv1.5 channel, including wild type and mutant Kv1.5 channel polypeptides, polynucleotides, and vectors and cells comprising the same.

Any Kv1.5 polypeptide may be used according to aspects of the present invention. While in certain embodiments, the Kv1.5 polypeptide is the wild type human Kv1.5 polypeptide having a sequence set forth in SEQ ID NO:1, Kv1.5 human homologs and homologs from other species can also be used. In addition, functional variants may also be used instead of or in addition to the wild type Kv1.5 polypeptide. Functional variants, as defined herein, are Kv1.5 polypeptide variants that retain the same or similar functional activity as a wild type Kv1.5 polypeptide, e.g., at least 75%, at least 80%, at least 90%, or at least 95% of the ion channel functional activity of wild type Kv1.5 polypeptide, as determined by an assay described herein. Kv1.5 polypeptide variants are polypeptides having at least 75%, at least 80%, at least 90%, or at least 95% sequence identity with a wild type Kv1.5 polypeptide, e.g., the Kv1.5 polypeptide of SEQ ID NO:1.

When comparing polypeptide or polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions (i.e., amino acid residues or nucleotides), usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., Unified Approach to Alignment and Phylogenes, pp. 626-645 (1990); Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Saitou, N. Nei, M., Mol. Biol. Evol. 4:406-425 (1987); Sneath, P.H.A. and Sokal, R. R., Numerical Taxonomy—the Principles and Practice of Numerical Taxonomy, Freeman Press, San Francisco, Calif. (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity for the polypeptides and polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

The present invention also includes mutants of wild type Kv1.5 polypeptides. As used herein, a mutant Kv1.5 polypeptide refers to a Kv1.5 polypeptide variant comprising one or more amino acid residue additions, deletions, or substitutions (collectively referred to as mutations). The one or more mutation may occur at any location within a Kv1.5 polypeptide. In particular embodiments, a mutation is present in the selectivity filter, deep pore, or S6 region of a Kv1.5 polypeptide. For ease of reference, the specific mutations described herein are defined with respect to the human Kv1.5 polypeptide having the sequence set forth in SEQ ID NO:1. However, it is understood that comparable mutations can be made in corresponding regions or residues of other Kv1.5 polypeptides, including homologs and functional variants.

In particular embodiments, a Kv1.5 mutant comprises a mutation such that the resulting mutant Kv1.5 ion channel is affected differently by an ion channel modulating compound than the wild type Kv1.5 ion channel. In certain embodiments, the resulting mutant Kv1.5 ion channel is differentially affected by different ion modulating compounds. For example, in one embodiment, the resulting mutant Kv1.5 ion channel is affected by vernakalant or flecamide, but not both. In related embodiments, the difference is more subtle, with the resulting mutant Kv1.5 ion channel being affected to a greater or lesser degree by one ion channel modulating compound as compared to another.

The effect of an ion channel modulating compound on a wild type or mutant Kv1.5 ion channel may be determined functionally, e.g., by measuring the compounds ability to block the channel. It may also be determined based upon the ability of the ion channel modulating compound to bind to the Kv1.5 ion channel. In particular embodiments, the block is a reduction in activity to less than 90%, less than 80%, less than 70%, less than 60%, less than 50%, less than 40%, less than 30%, less than 20%, or less than 10% the activity of the wild type Kv1.5 ion channel.

In particular embodiments, a mutant Kv1.5 polypeptide comprises a mutation, e.g., an amino acid substitution, in an amino acid residue corresponding to amino acid residue 479, 480, 502, 505, or 508, 510, 512, 516, or a region consisting of amino acid residues 500 to 512 or 509-531 of SEQ ID NO:1. In one preferred embodiment, a mutant Kv1.5 polypeptide comprises a mutation, e.g., an amino acid substitution at amino acid residue 502 of SEQ ID NO:1. Examples of other mutations that may be present in Kv1.5 mutants of the present invention are shown in Table I and Table II. In particular embodiments, a mutant Kv1.5 polypeptide contains or comprises 1, 2, 3, or more mutations.

Amino acid substitutions present in mutant Kv1.5 polypeptides may include the replacement of any amino acid residue with a different amino acid residue, including both naturally-occurring and artificial amino acids. In particular embodiments, the different amino acid residue is hydrophobic, while in other embodiments, it is hydrophilic. In other embodiments, the mutation results in the replacement of an amino acid residue with a different amino acid residue of greater or lesser molecular weight or size. In further embodiments, the substitution resulted in an increased ability or reduced potential for cation-π interactions in the Kv1.5 polypeptide.

In certain embodiments, polypeptides and polynucleotides are isolated. An “isolated” polypeptide or polynucleotide is one that is removed from its original environment. For example, a naturally-occurring protein or polypeptide is isolated if it is separated from some or all of the coexisting materials in the natural system. Preferably, such polypeptides and polynucleotides are also purified, e.g., are at least about 90% pure, more preferably at least about 95% pure and most preferably at least about 99% pure.

The present invention further includes polynucleotides that encode any of the Kv1.5 variant, functional variant, or mutant polypeptides described herein. Due to the degeneracy of the genetic code, several different polynucleotides may encode a single polypeptide. A polynucleotide sequence that encodes for the wild type human Kv1.5 polypeptide is shown in SEQ ID NO:2.

Kv1.5 mutant polypeptides may be readily produced using routine molecular biology techniques. For example, a mutagenesis approach, such as site-directed mutagenesis, may be employed for the preparation of mutants or variants described herein. By this approach, specific modifications in a polypeptide sequence can be made through mutagenesis of the underlying polynucleotides that encode them, e.g., the coding region of the polynucleotide that encodes human Kv1.5 polypeptide provided in SEQ ID NO:2. These techniques provide a straightforward approach to prepare and test sequence variants, for example, incorporating one or more of the foregoing considerations, by introducing one or more nucleotide sequence changes into the polynucleotide that encodes the mutant or variant polypeptide.

The preparation of sequence variants using site-directed mutagenesis provides a means of producing potentially useful species and is not meant to be limiting, as there are other ways in which sequence variants of Kv1.5 polypeptides and polynucleotide sequences encoding them may be obtained. For example, recombinant vectors encoding the desired sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982.

Polynucleotide segments or fragments encoding the polypeptides of the present invention may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202, by introducing selected sequences into recombinant vectors for recombinant production, and by other recombinant DNA techniques generally known to those of skill in the art of molecular biology (see for example, Current Protocols in Molecular Biology, John Wiley and Sons, NY, N.Y.).

In certain embodiments, wild type and mutant Kv1.5 polypeptides are produced by recombinant expression of a wild type or mutant Kv1.5 cDNA sequence, including the human cDNA sequence provided in SEQ ID NO:2. However, it will be appreciated by those of ordinary skill in the art that, as a result of the degeneracy of the genetic code, there are many nucleotide sequences that encode a particular polypeptide of interest. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Wild type and mutant Kv1.5 polypeptides may be recombinantly produced or expressed in a cell. The present invention includes cells comprising an exogenous Kv1.5 polypeptide and/or a polynucleotide that encodes for a mutant Kv1.5 polypeptide described herein. A polynucleotide sequence encoding the Kv1.5 polypeptide may be inserted into a cell, e.g., either transiently or stably integrated within the cell's genome. Expression of the Kv1.5 polypeptide may, therefore, be under the control of endogenous promoters and regulatory sequences within a cell, or under the control of exogenously introduced promoters. In particular embodiments, a Kv1.5 polypeptide is expressed in a cell from a suitable expression vector, which comprises promoter and other regulatory sequences, as well as a polynucleotide encoding the Kv1.5 polypeptide. A variety of suitable expression vectors are known and commercially available, and may be readily chosen and used. Typically, a vector is chosen based upon is suitability for the particular cell type into which it will be inserted for expression, such that the promoter and other regulatory elements are active within the cell type. Methods are available in the art for constructing vectors containing sequences encoding a polypeptide of interest and appropriate transcriptional and translational control elements. These methods include in vitro recombinant DNA techniques, synthetic techniques, and in vivo genetic recombination. Such techniques are described, for example, in Amalfitano et al., 1998 J. Virol. 72:926-933; Hodges, et al., 2000 J Gene Med 2:250-259; Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y. Expression vectors may be introduced into cells by a variety of means known in the art, including, e.g., transformation, transfection, or infection.

The Kv1.5 polypeptides of the present invention may be expressed in a variety of different host cells, including, e.g., microorganisms such as bacteria transformed with recombinant bacteriophage, plasmid, or cosmid DNA vectors; yeast transformed with yeast vectors; insect cell systems infected with virus vectors (e.g., baculovirus); plant cell systems transformed with virus vectors (e.g., cauliflower mosaic virus, CaMV; tobacco mosaic virus, TMV) or with bacterial vectors (e.g., Ti or pBR322 plasmids); or animal cell systems. Expression may be confirmed using any of a variety of protocols for detecting and measuring the expression of polynucleotide-encoded products (e.g., Kv1.5 polypeptides), using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

In particular embodiments, a host cell strain may be chosen for its ability to modulate the expression of the inserted sequences or to process the expressed protein in the desired fashion. For example, it is important for the host cell system to express the Kv1.5 polypeptides so that they will form an ion channel. In a related manner, it may be important the host cell does not express any endogenous ion channels, or any Kv1.5 ion channels in particular, so that the effects of the Kv1.5 mutations are not masked. In particular embodiments, it is desired to use a mammalian cell expression system, to enhance appropriate post-translational modification of mammalian Kv1.5 polypeptides. Such modifications of the polypeptide include, but are not limited to, acetylation, carboxylation. glycosylation, phosphorylation, lipidation, and acylation. In specific embodiments, mammalian host cells, such as CHO, COS, HeLa, MDCK, HEK293, and W138, having specific cellular machinery and characteristic mechanisms for such post-translational activities, are used to ensure the correct modification and processing of the exogenously introduced Kv1.5 polypeptide.

### Ion Channel Modulating Compounds and Methods of Identifying Same

As described herein, the present invention identifies specific mutations in Kv1.5 polypeptides that may be used to distinguish between different ion channel modulating compounds. Thus, the present invention allows the identification of ion channel modulating compounds, including antiarrhythmic agents, having one or more desired properties, such as high potency, atrial-selectivity, preferentially active on K⁺ or Na⁺ channels, or reduced association with ventricular proarrhythmias, such as Torsades de Pointes. Thus, the present invention provides such compounds having these and other desired properties.

The present invention includes methods of identifying ion channel modulating compounds based upon their interaction with Kv1.5 ion channels. These interactions may be determined in a variety of manners, including the binding of a compound to Kv1.5 channels or mutant Kv1.5 channels, or the ability of the compound to affect the activity of a Kv1.5 channel or mutant Kv1.5 channel. As described herein, different ion channels are differentially affected by different mutations in the Kv1.5 polypeptide, and these differences may be used to identify ion channel modulating compounds having the advantageous properties described herein. In particular embodiments, these methods are used to identifying compounds that preferentially bind to the same amino acid residues of Kv1.5 as bound by vernakalant.

In particular embodiments, methods of the present invention comprise comparing the ability of a candidate ion channel modulating compound to block or bind to wild type Kv1.5 ion channels to its ability to block or bind to one or more mutant Kv1.5 ion channels. As used herein, the term mutant Kv1.5 ion channel refers to ion channels comprising mutant Kv1.5 polypeptides. Typically, a mutant Kv1.5 ion channel will include only one type of Kv1.5 mutant polypeptide. In particular embodiments, these methods may also include identifying a candidate ion channel modulating compound that blocks or binds to a wild type Kv1.5 ion channel.

In general, methods of the present invention are practiced using cells expressing exogenously introduced Kv1.5 polypeptides, so as to express functional Kv1.5 ion channels. These polypeptides may be a wild type Kv1.5 polypeptide or a mutant form of a Kv1.5 polypeptide. Although more than one Kv1.5 polypeptide may be expressed in one cell, the methods of the invention are typically practiced using cells that express only one Kv1.5 polypeptide or mutant thereof. These cells are contacted with a candidate ion channel modulating compound, and the effect of the compound on the expressed Kv1.5 is channel is determined. In certain embodiments, the effect is determined functionally, e.g., by measuring an activity of the ion channel, or, alternatively, the binding of the compound to the Kv1.5 ion channel may be determined. A candidate ion channel modulating compound may be contacted with cells expressing one mutant Kv1.5 ion channel, or it may be contacted separately with cells expressing different mutant Kv1.5 ion channels, thereby generating a profile of the candidate compound's interaction with Kv1.5. In one embodiment, methods of the present invention are practiced in microtitre dishes, with different wells comprising different cells, each well containing cells comprising a different Kv1.5 wild type or mutant polypeptide. In another embodiment, different wells comprise different candidate ion channel modulating compounds. Thus, methods of the invention may be practiced using high throughput analysis.

In one embodiment, ion channel modulating compounds are identified based upon a preferred profile with respect to the Kv1.5 mutants. In particular embodiments, this profile is similar to the profile produced by vernakalant, in that mutations that alter vernakalant block of Kv1.5 ion channels also alter the Kv1.5 ion channel block produced by the candidate ion channel modulating compound. In one embodiment, mutations that enhance vernakalant block or binding also enhance block or binding by the candidate ion channel modulating compound. In a related embodiment, mutations that decrease block or binding by vernakalant also decrease block or binding by the candidate ion channel modulating compound.

Thus, in one embodiment, an ion channel modulating compound is identified when it blocks or binds a wild type Kv1.5 channel but the block or binding is reduced when the Kv1.5 polypeptide comprises a mutation in an amino acid residue corresponding to amino acid residue 480, or a region consisting of amino acid residues 500 to 508 of SEQ ID NO:1. In one particular embodiment, an ion channel modulating compound is identified when the block or binding is reduced at least 5-fold when the Kv1.5 polypeptide comprises a mutation in an amino acid residue corresponding to amino acid residue 502 of SEQ ID NO:1. In another particular embodiment, an ion channel modulating compound is identified when the block or binding is reduced when the Kv1.5 polypeptide comprises a mutation in an amino acid residue corresponding to an amino acid residue selected from 480, 500, 502, 505, and 508 of SEQ ID NO:1.

In another embodiment, an ion channel modulating compound is identified when it blocks or binds a wild type Kv1.5 channel and the block is increased when the Kv1.5 polypeptide comprises a mutation in an amino acid residue corresponding to amino acid residue 479 or 501 of SEQ ID NO:1.

In a further embodiment, an ion channel modulating compound is identified when its ability to block a mutant Kv1.5 ion channel is affected by any mutation as described above, but the block or binding is not significantly reduced when the Kv1.5 polypeptide comprises a mutation in a region corresponding to amino acid residues 509-531 of SEQ ID NO:1. In a particular embodiment, the block or binding is not significantly reduced when the Kv1.5 polypeptide comprises a mutation in an amino acid residue corresponding to amino acid 510, 512, or 516 of SEQ ID NO:1.

The ability of a candidate ion channel modulating compound to affect a Kv1.5 ion channel may be functionally determined using techniques known in the art for measuring ion channel activity. For example, in one embodiment, ion channel activity is determined by whole-cell current recording in a superfusion chamber using adherent cells plated on a coverslip, as described in Example 1. The ability of a candidate compound to block the ion channel can be determined by adding escalating concentrations of the compound to the bath solution, and concentrations approximately equal to the 50% inhibitory value for a compound on the wild type Kv1.5 ion channel can be determined. These wild type Kv1.5 IC₅₀ values can then serve as references used to compare the effect of various mutations on a compounds ability to block.

In particular embodiments, a decreased ability of a candidate compound to block a mutant Kv1.5 ion channel (as compared to a wild type Kv1.5 ion channel) is identified when the IC₅₀ determined using cells expressing the mutant Kv1.5 ion channel is at least 2-fold, at least 5-fold, at least 10-fold, at least 15-fold, at least 20-fold, or at least 25-fold higher than the IC₅₀ determined using cells expressing wild-type Kv1.5 ion channel. Similarly, an increased ability of a candidate compound to block a mutant Kv1.5 ion channel is identified when the IC₅₀ determined using cells expressing the mutant Kv1.5 ion channel is less than 90%, less than 80%, less than 70%, less than 60%, less than 50%, less than 40%, less than 30%, less than 20%, or less than 10% of the IC₅₀ determined using cells expressing wild-type Kv1.5 ion channel.

In other embodiments, an increased ability of a candidate compounds to block a mutant Kv1.5 ion channel (as compared to a wild type Kv1.5 ion channel) is identified when the fractional block determined using cells expressing the mutant Kv1.5 ion channel is at least 2-fold, at least 5-fold, at least 10-fold, at least 15-fold, at least 20-fold, or at least 25-fold higher than the fractional block determined using cells expressing the wild type Kv1.5 ion channel. Similarly, a decreased ability of a candidate compound to block a mutant Kv1.5 ion channel is identified when the IC₅₀ determined using cells expressing the mutant Kv1.5 ion channel is less than 90%, less than 80%, less than 70%, less than 60%, less than 50%, less than 40%, less than 30%, less than 20%, or less than 10% of the IC₅₀ determined using cells expressing wild-type Kv1.5 ion channel.

The ability of a candidate ion channel modulating compound to bind to wild type or mutant Kv1.5 ion channels may be determined using methods known in the art, including, e.g., using FACS analysis or FMAT™ analysis and instrumentation (Applied Biosystems, Foster City, Calif.). FMAT™ is a fluorescence macro-confocal platform for high-throughput screening, which mix-and-read, non-radioactive assays using live cells or beads. For example, cells and a candidate ion channel modulating compound may be labeled with different fluorescent tags, and the ability of the candidate molecule to bind the Kv1.5 ion channel expressed by the cells determined by the FACS identification of cells associated with both fluorescent labels.

Candidate ion channel modulating compounds include both known ion channel modulating compounds, as well as ion channel modulating compounds that have not yet been identified. Methods of the present invention may be practiced using known compounds, or a library of compounds may be screened against wild type Kv1.5 ion channels and/or different mutants thereof.

In one embodiment, an ion channel modulating compound is identified by screening libraries of molecules or chemical compounds, e.g., small molecules. Such libraries and methods of screening the same are known in the art and include: biological libraries, natural products libraries, spatially addressable parallel solid phase or solution phase libraries, synthetic library methods requiring deconvolution, the ‘one-bead one-compound’ library method, and synthetic library methods using affinity chromatography selection. The biological library approach is largely limited to polypeptide libraries, while the other four approaches are applicable to polypeptide, non-peptide oligomer or small molecule libraries of compounds. See Lam, K. S. (1997) Anticancer Drug Des. 12:145. In certain embodiments, screening of libraries is performed using an array or microarray, which permits the testing of multiple compounds, e.g., small molecules, polypeptides, or antibodies) simultaneously. In particular embodiments, screening is high throughput screening.

In one embodiment, inhibitors are identified using Automated Ligand Identification System (referred to herein as “ALIS”). See, e.g., U.S. Pat. Nos. 6,721,665, 6,714,875, 6,694,267, 6,691,046, 6,581,013, 6,207,861, and 6,147,344. ALIS is a high-throughput technique for the identification of small molecules that bind to proteins of interest (e.g., Kv1.5 polypeptides). Small molecules found to bind tightly to a protein can then be tested for their ability to inhibit the biochemical activity of that protein or associated ion channel.

In certain embodiments, cells expressing wild type Kv1.5 or a mutant Kv1.5 are mixed with pools of small molecules. Preferably, more than 1,000 pools are used, more preferably more than 2,000 pools are used, more preferably more than 3,000 pools are used, or more preferably, more than 10,000 pools are used. Each pool contains approximately, 1,000 compounds, more preferably approximately 2,500 compounds, or more preferably approximately 5,000 compounds that are ‘mass encoded,’ meaning that their precise molecular structure can be determined using only their mass and knowledge of the chemical library. In one embodiment, the small molecules and cells are mixed together and allowed to come to equilibrium (they are incubated together for 30 minutes at 37 degrees C.). The cell are then washed and bound small molecules analyzed using mass spectroscopic analysis to determine the masses of all small molecules found to bind the cells. Measurement of these masses allows for the rapid determination of the molecular structures of the small molecules. In another embodiment, the small molecules and cells are mixed together and whole-cell patch clamp analysis performed to determine the effect of the compounds on blocking the expressed ion channel. Limiting dilution may be performed to identify a single small molecule that blocks wild type Kv1.5 ion channels but whose block is affected by a Kv1.5 mutation as described above, thereby identifying this molecule as an ion channel modulating compound having advantageous properties according to the present invention.

### EXAMPLES

### Example 1

Vernakalant (RSD1235) is an ion channel modulating compound shown to convert atrial fibrillation rapidly and safely in patients (Roy et al., 2004). Here, the molecular mechanisms of interaction of vernakalant with the inner pore of the Kv1.5 channel were compared with that of the Class IC agent flecamide. Initial experiments showed that vernakalant blocks activated channels and must vacate the inner vestibule in order for the channel to close, and thus mutations were made at the base of the selectivity filter and in S6, by also drawing on studies of other Kv1.5-selective agents. Block by vernakalant or flecamide was assessed by changes in whole cell current in transiently transfected HEK293 cells after application of drug. Results from the mutational scan identified several highly conserved amino acids, T479, T480, I502, V505 and I508, as important residues for affecting block by both compounds. In general, mutations in S6 reduced the IC₅₀ for block by vernakalant, with I502A causing an extremely local 25-fold decrease in potency. Specific changes in the voltage-dependence of block with I502A supported the crucial role of this position in vernakalant potency, and highlighted a difference with flecamide which showed potency effects more distributed along S6. A homology model of the pore region of Kv1.5 predicted that of these residues only T479, T480, V505 and I508 are potentially accessible for direct interaction, and that mutation at additional sites studied may therefore affect block through allosteric mechanisms. For some of the mutations, the direction of changes in IC₅₀ were opposite for vernakalant and flecamide, highlighting differences in the forces that drive drug-channel interactions.

### Materials and Methods

Cell Preparation

Stable lines of HEK293 cells expressing Kv1.5, or transient transfection of mutant Kv1.5 channels were used in all experiments. Primers to generate mutant channels were synthesized by Integrated DNA Technologies, Inc. (Coralville, Iowa, USA), and mutants were generated using the Stratagene Quikchange kit (Stratagene, Calif., USA). The presence of the mutation was confirmed by DNA sequencing, and because of the size of the WT Kv1.5 clone, only the sequenced region harboring the targeted nucleotides was subcloned as a fragment back into full-length Kv1.5. Transient transfections were performed with HEK 293 cells plated at 20-30% confluency on sterile coverslips in 25 mm Petri dishes one day prior to transfection. 0.1-4 μg of ion channel DNA was incubated with 1 μg of eGFP DNA (to enable detection of transfected cells) and 2 μl of lipofectamine 2000 (Invitrogen) in 100 μl of Opti-MEM. This was added to the cells for overnight incubation after washing the media with 900 μl of MEM with 10% FBS.

The cells were grown in Minimal Essential Medium (MEM) at 37° C. in an air/5% CO₂ incubator. Media contained 10% bovine serum and 0.5 mg/ml geneticin for stable lines. On the day before recording, stably-expressing cells were washed with MEM, treated with trypsin/EGTA for one minute and plated on 25 mm² coverslips. All cell culture supplies were obtained from Invitrogen (Mississauga, ON, Canada).

### Drugs and Solutions

Control bath solution contained (in mM): 5 KCl, 135 NaCl, 2.8 sodium acetate, 1 MgCl₂, 10 HEPES and 1 CaCl₂, adjusted to pH 7.4 with NaOH. Patch pipettes contained (in mM): 130 KCl, 5 EGTA, 1 MgCl₂, 10 HEPES, 4 Na₂ATP, and 0.1 GTP, adjusted to pH 7.2 with KOH. All chemicals used to make solutions were obtained from Sigma-Aldrich (Mississauga, ON, Canada). Vernakalant (also known as RSD1235; 3-Pyrrolidinol, 1-[(1R,2R)-2-[2-(3,4-dimethoxyphenyl)ethoxy]cyclohexyl]-,hydrochloride, (3R))) was synthesized by Cardiome Pharma Corp. (Vancouver, BC, Canada) and prepared as a stock solution (50 mM) in H₂O. Flecamide (Sigma-Aldrich, lot number 094K4057) was prepared as a stock solution (100 mM) in 100% DMSO. DMSO concentrations for either drug never exceeded 0.1% v/v in the final experimental solutions.

Whole-Cell Patch-Clamp Recordings

Coverslips with adherent cells plated on the surface were placed in a superfusion chamber (volume 300 μl) containing the control bath solution at 22° C. Whole-cell current recording and analysis were carried out using an Axopatch 200B amplifier and pClamp10 software (Axon Instruments, CA, USA). Patch electrodes were pulled from thin-walled borosilicate glass (World Precision Instruments, FL, USA) on a horizontal micropipette puller (Sutter Instruments, CA, USA). Electrodes had resistances of 1.0-3.0 MΩ when filled with control filling solution. Analogue capacity compensation and 80% series resistance compensation were used during whole cell measurements. Membrane potentials were not corrected for junction potentials that arise between the pipette and bath solution. For Kv1.5 current recordings, a holding potential of −80 mV was used. Data were sampled at 10-20 kHz and filtered at 5 to 10 kHz. To assess drug block, half-log escalating concentrations of drug were added to the flowing bath solution and current traces were recorded with 400 ms depolarizing pulses to +60 mV at a frequency of 0.1 Hz. Concentrations approximately equal to the 50% inhibitory value for each drug on the WT channel were used in the initial investigation of vernakalant and flecamide actions on mutated channels.

Data Analysis

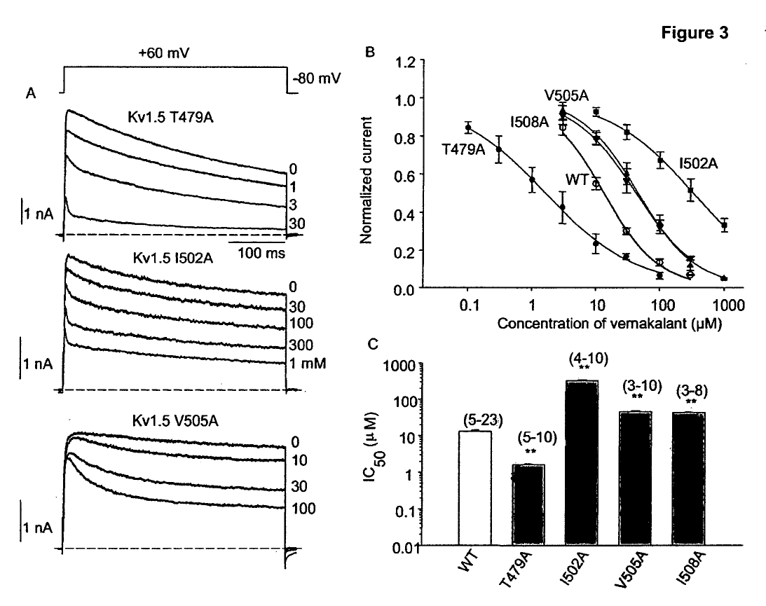

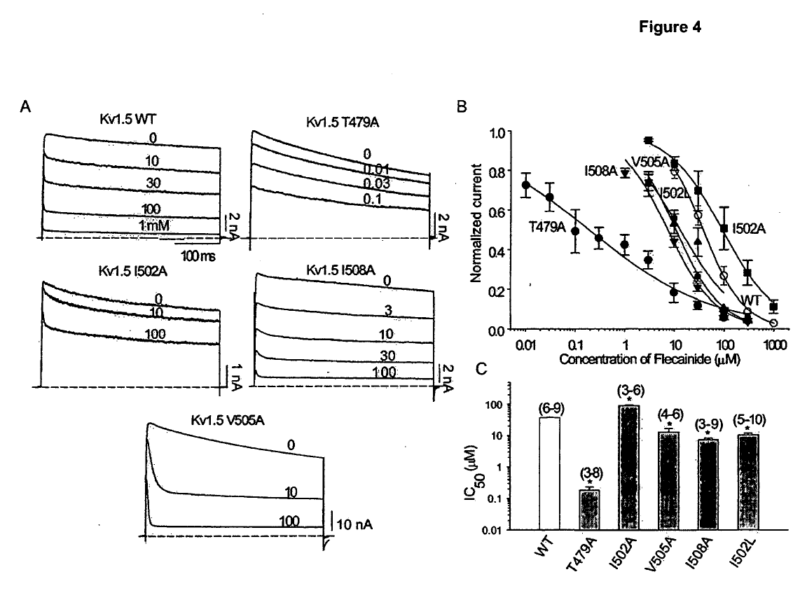

Data are represented as mean±SE unless otherwise specified. For significance of differences, * represents p<0.05, ** p<0.01; statistical analysis was conducted using Student's t-test (unpaired). Clampfit software (Axon Instruments, CA, USA) was used to analyze current traces. Data from each test pulse (currents in the presence of drug) were normalized to control test pulse currents obtained before drug exposure. For drug potency studies of the mutant Kv1.5 channels in the presence of vernakalant or flecamide (FIGS. 3, 4), currents in the presence of different concentrations of drug were normalized to the current in control and used to generate concentration-response curves for changes in steady state Kv1.5 current. The resulting concentration-response relations were computer-fitted to the Hill equation:

i=1/[1+(IC₅₀/[D]ⁿ)]

where i is the fractional current block (i=1−I_{drug}/I_{control}) at drug concentration [D]; IC₅₀ is the concentration producing half-maximal inhibition; and n is the Hill coefficient.

The voltage dependence of block for vernakalant and flecamide was determined by the calculation of the fractional block (f=1−I_{drug}/I_{control}) at the potentials in the range of full channel opening (between +10 to +60 mV) and fitting the data to the Woodhull equation:

f=[D]/([D]+K_{d}*e^{-δzFE/RT})

where F, R, z and T have their usual meanings, δ represents the fractional electrical distance, i.e., the fraction of the transmembrane electrical field sensed by a single charge at the receptor site. K_{d}* represents the binding affinity at the reference voltage (0 mV). The current amplitude (I_{drug} and I_{control}) was measured at the end of a 400 ms depolarization.

Homology Model

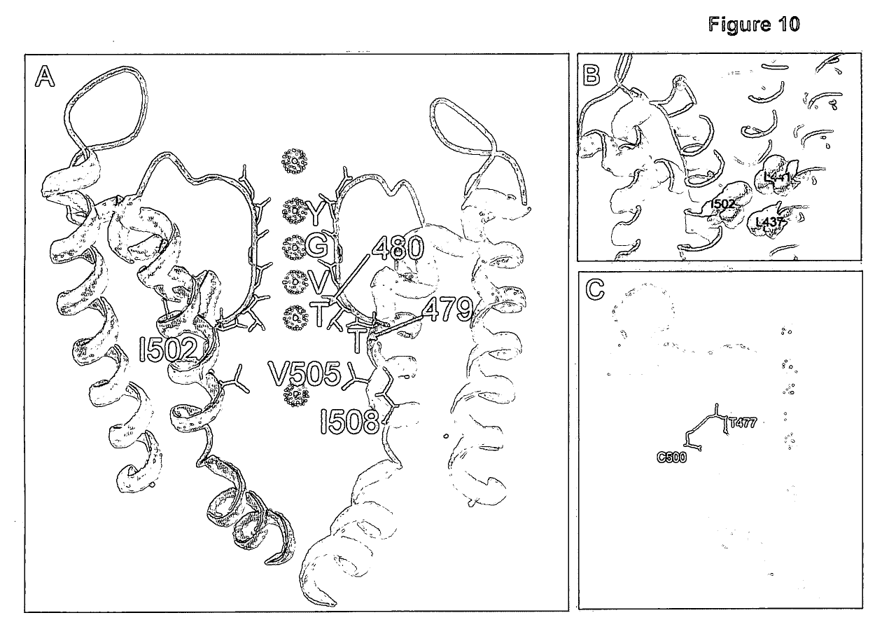

The Kv1.5 homology model was generated using “First Approach Mode” of SWISS-MODEL (http://swissmodel.expasy.org) and the three-dimensional structure of rKv1.2 (Protein Data file 2A79; (Long et al., 2005), which is believed to represent the open state of the channel. rKv1.2 and hKv1.5 show 100% Identity in S5 and S6 and 88% identity in the pore loop. FIG. 10 was generated using DeepView Swiss-PdbViewer (http://swissmodel.expasy.org/spdbv) and Adobe Photoshop software.

### Results

Delayed Closing of Kv1.5 Channels in the Presence of Vernakalant at the Inner Pore

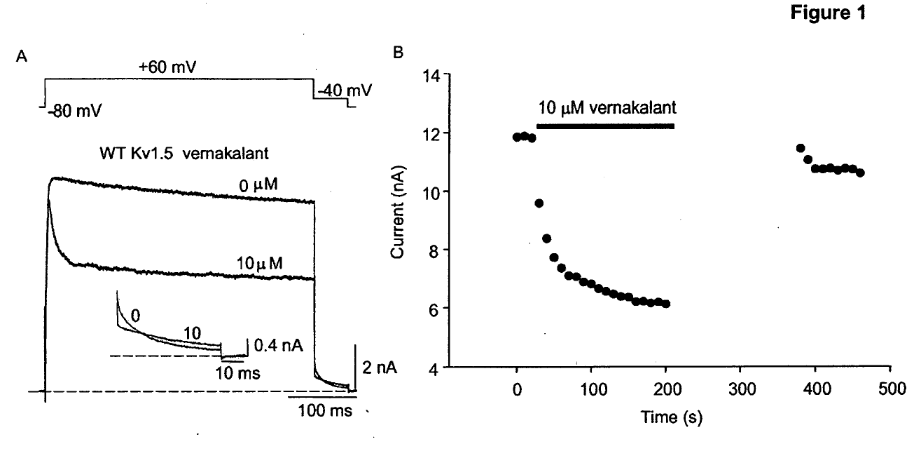

Previous experiments suggested that block of Kv1.5 by vernakalant was mediated after channel activation, as vernakalant caused a relatively rapid onset of block of channel current upon depolarization (Fedida et al., 2005), but little evidence of resting or ‘tonic’ block of the channel. In initial experiments aimed at localizing the site of binding of vernakalant within Kv1.5, the ability of the drug to affect activation gate closure upon repolarization, after channel opening was examined (FIG. 1). Control currents in FIG. 1A activated rapidly upon depolarization to +60 mV and showed only limited inactivation during the 400 ms depolarization shown here. During repolarization to −40 mV, a deactivation tail current was observed that reflects channel closure. In the presence of 10 μM vernakalant, rapid block was apparent after channel opening and a steady-state current level was rapidly reached. Upon repolarization, the tail current amplitude was initially less than in control, but decayed more slowly and crossed over the control tracing as it declined to the baseline current level (see inset panel). The result suggested that vernakalant delayed closing of Kv1.5, and perhaps that the channels could not close while vernakalant was bound, but required drug expulsion from the inner vestibule before that could happen.

This possibility was tested in the experiment shown in FIG. 1B. Here, after equilibration at 0.1 Hz in control solutions, the cell was exposed to vernakalant was shown. This led to a progressive reduction of steady-state current. At 200 s, the cell was rested for 3 min and vernakalant was washed from the bath. When the current was activated again by depolarizing clamp pulses, the current level was immediately restored to the control level. This suggested that vernakalant was not trapped in the closed channels while drug was washed from the bath, but rather that, as channels closed, vernakalant was slowly expelled from the inner pore. This slow expulsion is the cause of the slowed tail current observed in the presence of vernakalant (FIG. 1A).

Alanine Scan of S6: Block by Vernakalant

The data in FIG. 1 suggested that the S6 domain that lines the inner vestibule of the channel and the deep pore near the selectivity filter were likely areas where vernakalant bound to Kv1.5. These regions were targeted for mutational analysis of block by replacement of WT residues with alanines wherever possible, and valines if the WT residue was an alanine, and leucine, valine, or glycine if there was an expression problem with alanine. A summary of the mutants used in the present experiments is shown in Table I, and includes those for which current could not be recorded.

TABLE I
Summary of Fractional Block and IC₅₀ Values for Block by
Vernakalant for Wild-type and Mutant Kv1.5 Channels.

| | Fractional | | | _{IC50} ± SEM | Hill | Fold | |
|---|---|---|---|---|---|---|---|
| | Block ± SEM | Significance | n | (μM) | coefficient | change | n |
| WT | 0.45 ± 0.03 | | 23 | 13.3 ± 0.93 | 1.00 | | 5-23 |
| T479A | 0.77 ± 0.05 | | 9 | 1.63 ± 0.09 | 0.6 | 8.31 | 5-10 |
| T479S | 0.23 ± 0.04 | ** | 7 | 41.02 ± 3.54 | 0.95 | 3.07 | 3-8 |
| T480A | 0.23 ± 0.04 | ** | 12 | 52.59 ± 7.85 | 0.88 | 3.94 | 3-12 |
| T480S | 0.25 ± 0.07 | * | 6 | 52.83 ± 12.46 | 0.64 | 3.96 | 4-7 |
| V481L | 0.36 ± 0.03 | | 6 | | | | |
| C500A | 0.21 ± 0.04 | ** | 10 | 45.63 ± 4.06 | 0.95 | 3.42 | 3-10 |
| A501V | 0.69 ± 0.04 | ** | 8 | 3.49 ± 0.19 | 0.85 | 3.81 | 4-8 |
| I502A | 0.07 ± 0.03 | ** | 9 | 329.26 ± 18.97 | 0.65 | 24.66 | 4-10 |
| I502L | 0.33 ± 0.07 | | 6 | 24.84 ± 3.14 | 0.96 | 1.86 | 3-6 |
| I502F | 0.10 ± 0.01 | ** | 4 | 149.29 ± 8.36 | 0.84 | 11.11 | 4 |
| I502K | DNE | | 6 | | | | |
| I502D | DNE | | 5 | | | | |
| V505A | 0.21 ± 0.04 | ** | 10 | 45.54 ± 2.56 | 0.95 | 3.41 | 3-10 |
| V505L | 0.22 ± 0.07 | * | 4 | 57.70 ± 4.63 | 0.77 | 4.32 | 3-5 |
| V505F | 0.27 ± 0.05 | * | 6 | 28.79 ± 0.79 | 0.96 | 2.16 | 5-6 |
| V505K | DNE | | 5 | | | | |
| V505D | DNE | | 5 | | | | |
| I508A | 0.21 ± 0.03 | ** | 8 | 42.44 ± 0.94 | 0.87 | 3.18 | 3-8 |
| I508L | 0.38 ± 0.05 | | 4 | 16.84 ± 0.48 | 1.00 | 1.26 | 3-4 |
| I508Y | 0.21 ± 0.03 | ** | 7 | 24.70 ± 0.63 | 1.46 | 1.85 | 4-7 |
| I508F | 0.94 ± 0.02 | ** | 5 | 0.61 ± 0.03 | 1.23 | 21.80 | 4-8 |
| I508K | DNE | | 5 | | | | |
| I508D | DNE | | 5 | | | | |
| A509G | 0.33 ± 0.07 | | 9 | | | | |
| A509V | DNE | | 12 | | | | |
| V512L | 0.56 ± 0.04 | | 9 | | | | |
| P532L | 0.26 ± 0.03 | ** | 16 | 32.02 ± 1.83 | 0.96 | 2.40 | 3-16 |

p < 0.05;
**p < 0.01;
DNE = Did not Express

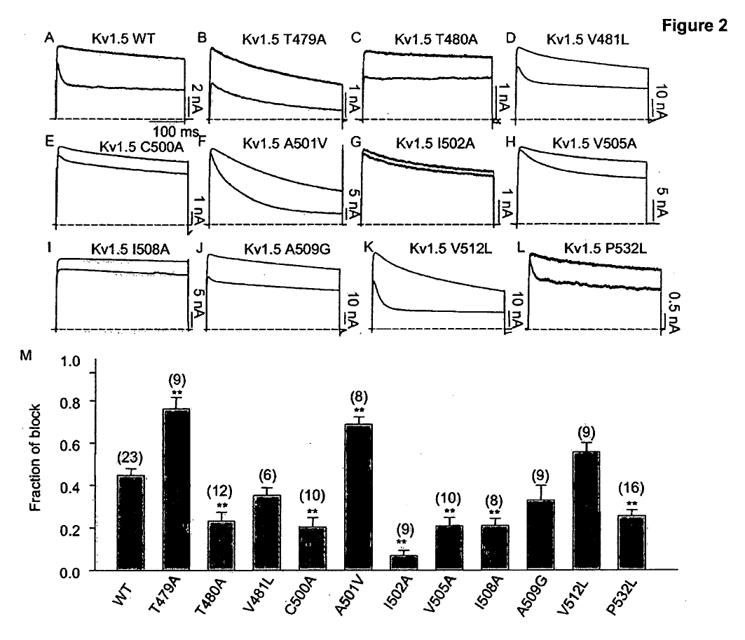

Channels transiently or stably expressed in HEK cells were voltage clamped with depolarizing pulses from −80 to +60 mV at 0.1 Hz, and allowed to equilibrate for 5 min before drugs were applied. Control currents in each panel of FIG. 2 show some differences between mutants. Most of the examples showed the rapidly activating, delayed rectifier current expected from Kv1.5 channels, with a minor degree of slow inactivation occurring during the 400 ms depolarizations used here. However, a few channels, Kv1.5 T479A, A501V, and V512L showed a definite increase in inactivation rate.

Initially, a scan of residues using 10 μM vernakalant was carried out to generally assess the importance of residues in this area. This concentration reduced currents significantly in almost all mutants studied, with an approximately 50% reduction of outward current in WT channels, as has been previously reported (Fedida et al., 2005), and reduced block in most other mutants with the least effect on the I502A mutant (FIG. 2G). It was noted that the three mutants with increased inactivation all showed greater block at 10 μM vernakalant than control, and this finding is clearly seen in the bar graph below which summarizes data from the S6 mutations (FIG. 2M). As well, T479 and 480 are located at the inner mouth of the selectivity filter and any changes that these mutations induced in K+ selectivity or permeation might be independent actions on drug block. As a result, the data from these mutants required special consideration when compared with the data from other mutations.

Significant reductions in block by 10 μM vernakalant appeared to be centred on I502, with a diminishing effect for mutations moving away from this site, disappearing distally along S6 by residue A509, and proximally by residues V481 and C500 at the base of the selectivity filter.

Full dose-response relationships for vernakalant (FIG. 3) were obtained for the reduced block mutants, I502A, V505A, and I508A, in comparison with a positive control mutation that increased block (T479A), and for a readily available comparative antiarrhythmic agent, flecamide (FIG. 4), whose block was also reported to be affected by mutations in the lower S6 (Herrera et al., 2005). Vernakalant data shown in FIG. 3 confirmed the 10 μM scan in FIG. 2, with increased potency for T479A, and with reduced potency for the other mutants. Original tracings are shown in FIG. 3A, and illustrate the actions of vernakalant at concentrations between 1 μM and 1 mM in the different mutants. Steady-state current block was plotted against drug concentration (FIG. 3B) and fitted with a Hill equation to obtain the IC₅₀ values for the different mutants, which are also shown in the bar graph below (FIG. 3C).

The most important effect was the reduction in potency for vernakalant centred at I502A which had an IC₅₀ of 329±19 μM (n=4-10, see Table I), compared with a control IC₅₀ of 13.4±0.9 μM (n=5-23), which is a 25-fold decrease in potency. V505A, I508A, T480A, and C500A showed lesser reductions in potency on Kv1.5 (Table I), of between 3- and 4-fold (Table I). Similar experiments were carried out for flecamide (FIG. 4A), and in qualitative agreement with previous oocyte data from the nearby residue in the selectivity filter, V481 (Herrera et al., 2005), the mutation of T479 to alanine increased potency of flecamide (FIGS. 4B, C), in this case 238-fold, from a control IC₅₀ of 38.1±1.1 μM (n=6-9, see Table II) to an IC₅₀ of 0.19±0.04 μM (n=3-8). I502A showed a diminished potency in the presence of flecamide, with an increase in IC₅₀ from 38.1 μM in control to an IC₅₀ of 92±5.5 μM (n=3-6), a 2.4-fold reduction in potency (FIGS. 4B, C). Clearly, the effect of this mutation on flecamide block was much less than on vernakalant block, and in a related area, insertion of alanines at V505 and I508 increased the potency for flecamide block 3-fold and 5.2-fold respectively (FIG. 4C), in contrast to the reduction in block of 3 to 4-fold observed for vernakalant (FIG. 3C).

TABLE II
Summary of IC₅₀ Values for Block by
Flecainide for Wild-type and Mutant
Kv1.5 Channels.

| | IC₅₀ ± SEM | | Fold | |
|---|---|---|---|---|
| | (μM) | Significance | Change | n |
| WT | 38.14 ± 1.06 | | | 6-9 |
| T479A | 0.19 ± 0.04 | *** | 200 | 3-8 |
| I502A | 92.04 ± 5.54 | *** | 2.43 | 3-6 |
| I502L | 10.75 ± 1.09 | *** | 3.55 | 5-10 |
| I502F | 164.49 ± 31.36 | *** | 4.13 | 3-6 |
| V505A | 13.076 ± 3.58 | *** | 2.92 | 4-6 |
| V505F | 4.27 ± 0.34 | *** | 8.93 | 4-6 |
| I508A | 7.39 ± 0.92 | *** | 5.15 | 3-9 |
| I508F | 74.71 ± 5.37 | *** | 1.96 | 4-9 |

*** p < 0.001

Further Examination of Other Amino Acid Substitutions in S6 on the Potency of Vernakalant and Flecamide Action

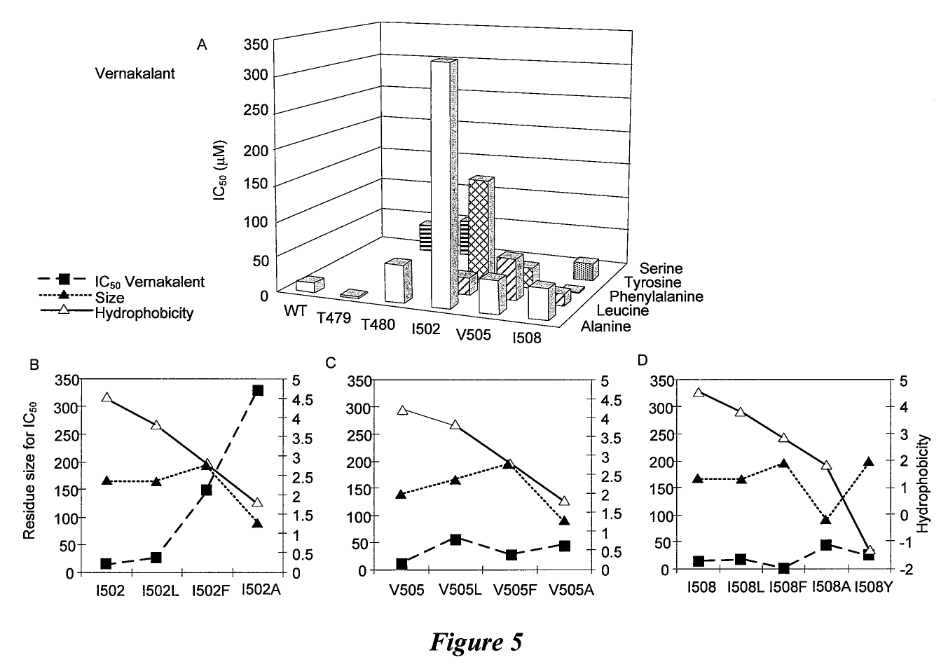

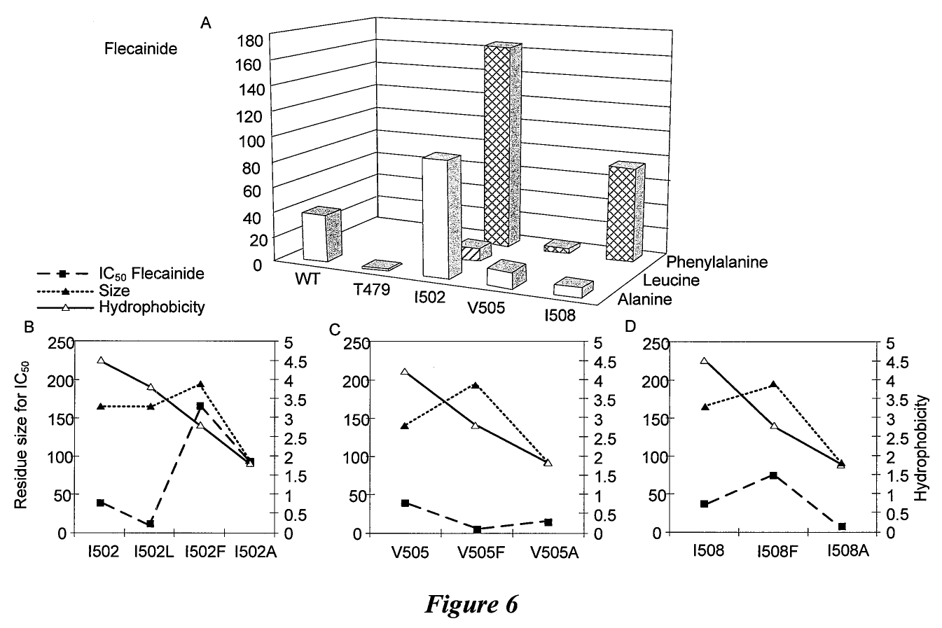

Given the obvious importance of I502 in the block by vernakalant, a series of other residues were substituted for isoleucine at this site, and close by, in order to alter the hydrophobicity, the residue size, and the potential for cation-π interactions in this region. The results for vernakalant are shown in FIG. 5, and comparative data for flecamide in FIG. 6 (see also Table I). For substitutions at I502, there was a clear modulation of vernakalant IC₅₀ that was inversely related to the hydrophobicity of the substitution made, with the native isoleucine conferring the most potent block, and alanine the least potent block. Overall, the potency series was I>L>F>A (FIG. 5B). At V505, a similar trend was seen, albeit less marked, and at I508, no such correlation was apparent (FIGS. 5B, 5C). In contrast, cation-π interactions appeared unimportant at I502, but become increasingly important moving to 505 and 508 (FIGS. 5A, 5C, 5D), such that the potency for vernakalant on I508F was 0.61 μM, the highest obtained in the present series of experiments (FIG. 5A and Table I). This result suggested that 1-stacking between aromatic groups of the drug and I508F confers high affinity binding. As well, at I508, a good correlation was apparent between vernakalant IC₅₀ and residue size, which suggested that increasing side-chain volume at this location caused steric hindrance (FIG. 5D).

In the case of flecamide, there was no obvious correlation with the hydrophobicity of the substitution at I502, V505, or I508 (FIGS. 5A-D). However, in contrast to vernakalant, residue size appeared to correlate linearly with flecamide IC₅₀ values at position 508 (FIG. 6D), and inversely with IC₅₀ at position 505. There was also the suggestion that size was important for flecamide IC₅₀ at position 502, where directional changes in potency matched the size change from residue to residue (FIG. 6B). Cation-x interactions appeared less important for flecamide than for vernakalant, or the geometric needs were more specific, i.e., the aromatic needs to be a specific distance from other sites of interaction. V505F seemed to be an exception to this, where the IC₅₀ value was 4.3 μM, compared with 38 μM in the WT when valine was present.

Voltage-Dependent Block of Kv1.5 by Vernakalant and Flecamide

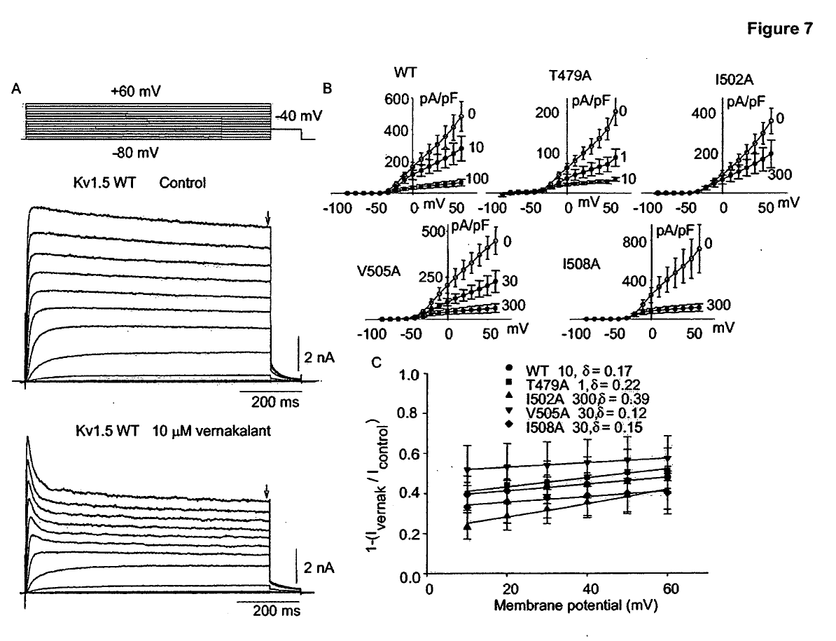

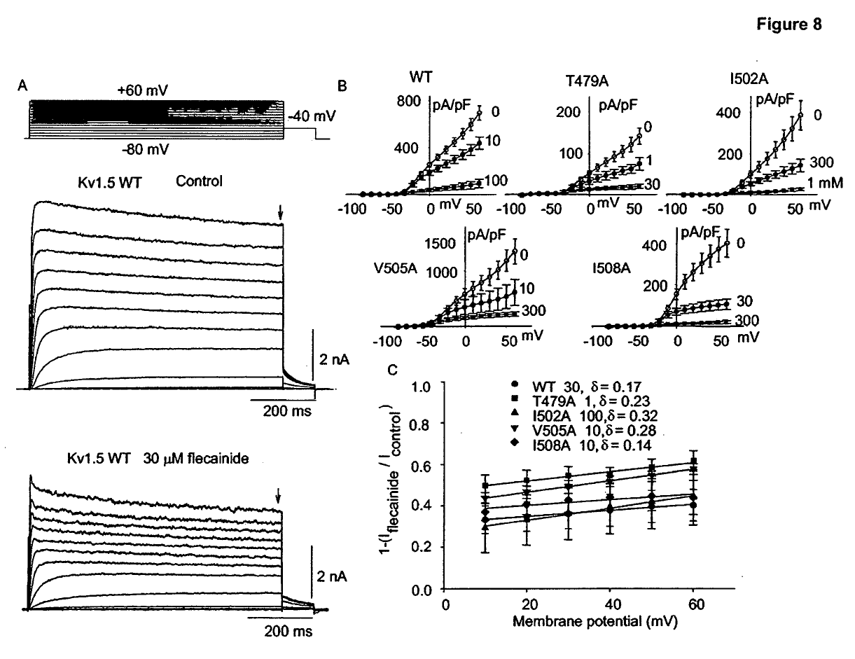

Electrophysiological evidence in support of I502 as a critical residue in the association of vernakalant with Kv1.5 channels was obtained from studies of the voltage-dependence of drug block. As before, comparative experiments were carried out between vernakalant and flecamide (FIGS. 7 and 8). In these experiments, cells were exposed to concentrations of vernakalant (FIG. 7) and flecamide (FIG. 8) near their IC₅₀s, and depolarized to a range of potential between −80 and +60 mV as illustrated by the protocol at the top of each figure. Current-voltage relationships were determined for WT and mutant channels (T479A, I502A, V505A, I508A) in the presence of either vernakalant of fleicanide (FIGS. 7B, 8B). The amount of block was measured at the end of the pulse at the time indicated by the arrow on each set of data records, and, after normalization to the control, current density was plotted as a function of the depolarization potential (FIGS. 7B, 8B). Data were then fit to a standard model of drug block to obtain the electrical distance of the blocking site into the electric field from the inside of the channel (Woodhull, 1973). In WT channels, the blocking site was located ˜17% of the electrical distance across the membrane (FIGS. 7C, 8C), and this was only perturbed in the presence of vernakalant in the I502A mutant, where analysis demonstrated a blocking site ˜40% of the electrical distance across the membrane. For flecamide, results were somewhat different. While I502A did increase the electrical distance to 32%, a significant increase in distance was also observed in V505A. In general, these results strongly support the inner S6 site for the actions of both vernakalant and flecamide, but suggest that vernakalant's site of action is very narrowly aligned with I502, whereas that of flecamide is more distributed over a couple of turns of the S6 helix from I502 to beyond V505.

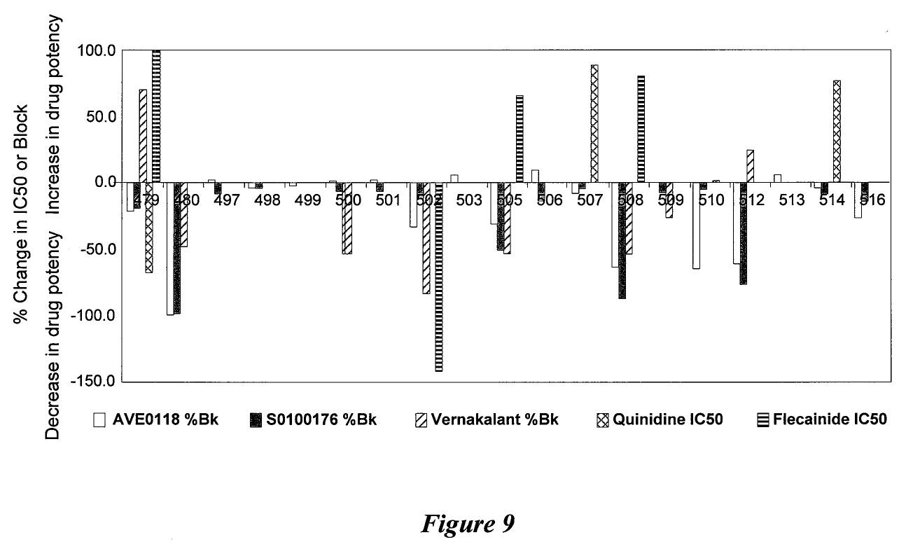

These differential effects of alanine substitution on the binding in the S6 region of the channel of vernakalant, flecamide, and some other antiarrythmic agents (including both Na⁺ channel and K⁺ channel blockers, data obtained from the literature (Yeola et al., 1996; Herrera et al., 2005; Decher et al., 2004; Decher et al., 2006)), are shown in summary form in FIG. 9. It can be seen that AVE0118, which has an apparent maximum molecular length of approximately 18 Å exhibited the most significant changes in IC₅₀ with mutations between I502 and V512, some four turns of the helix, with minor reductions in potency at V516. In contrast, the IC₅₀ for flecamide (molecular length 10 Å) was affected by mutations over a narrow range of residues more located in the deep pore. The atrial K⁺ channel blocker, S0100176 was affected by mutations in a similar way to AVE0118, but the extent of these effects was more limited, extending from V505 to V512, with only minor affects extending to V516, consistent with the differences in structure between these two drugs (Decher et al., 2006). There were also differential effects of mutations between drugs though to act primarily on Na⁺ channels, and those with more dominant K⁺ channel actions (FIG. 9). Flecamide and quinidine showed increased potency with mutations in the lower part of S6 between position 503 and 516, whereas those drugs that are more potent on K⁺ channels, including vernakalant, AVE0118, and S0100176, all showed decreases in potency with mutations throughout the same region (FIG. 9). However, unlike AVE0118, S0100176 and quinidine, T479A increased vernakalant sensitivity and flecamide sensitivity, suggesting a common interaction at this site.

Structural Consideration from Homology Modeling

Mutations at C500 and A501 both resulted in significant, though opposite, changes in IC₅₀ for vernakalant (Table I), and in contrast no differences were observed for AVE0118 and S0100176 (Decher et al., 2004; Decher et al., 2006) (FIG. 9). A homology model of Kv1.5 based on Kv1.2 (Long et al., 2005) was constructed, and this put the two residues at the base of the pore helix with a predicted H-bond between C500 and T477 in the adjacent helix (FIG. 10C). Mutation of the analogous residue in Kv2.1 (C393A) affected the stability of the open state and ion permeation without affecting external or internal TEA block (Liu and Joho, 1998). The A501V mutation had the most dramatic effects on inactivation and was one of the more sensitive mutants to vernakalant (FIG. 2 and Table I), and it is possible that disruption of the putative H-bond, with subsequent change of inactivation gating is responsible, at least in part, for the potency increase and the differential effect of vernakalant on this residue compared with A501.

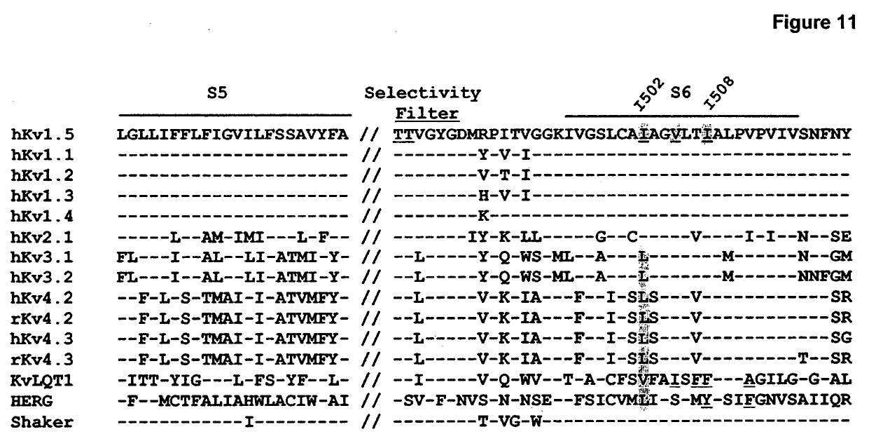

In the homology model of Kv1.5, I502 appears to point away from the internal cavity of the vestibule and towards two highly conserved leucines (L437 and L441; FIGS. 10A, 10B, 11) in an adjacent subunit (FIG. 10B), which makes it surprising that mutations at this site have such large effects on drug affinity. Given that potential interactions between I502 and the leucines in the adjacent subunit would involve hydrophobic interactions, changes in hydrophobicity at I502 might interfere with subunit packing and thus alter the binding site via an allosteric mechanism. Depending on the number of other interactions between the drug and the channel and the strength of these interactions, the changes around I502 may have greater or lesser affects. At this site, changes were more dramatic for vernakalant than AVE0118, which interacted over a greater number of residues within the channel (FIG. 9), consistent with its more extended configuration (approximately 18A).

### DISCUSSION

This study examined the binding of the atrial antiarrhythmic agent, vernakalant, to the Kv1.5 channel that underlies the rapidly-activating atrial K⁺ current, I_{Kur}. Previous work had suggested that channel activation was required for drug action (Fedida et al., 2005), and in support of this, as also described for AVE0118 (Decher et al., 2006), vernakalant was shown to slow channel closing by hindering movement of the deactivation gate, like a “foot-in-the-door” (FIG. 1). This was seen as a crossover of tail currents recorded in the presence and in the absence of drug. In this condition, the channel can only close after unbinding of the drug. This initial observation, combined with the work of others on potential binding sites for antiarrhythmic drugs within the inner vestibule of a variety of K⁺ channels allowed the scope of mutational analysis of the drug binding site to be focused on the inner vestibule and deep pore of the channel.

Findings from the Alanine Scan

An alanine scan was carried out of residues in the deep pore/pore helix (T449-V481) and lower S6 (C500-V512), though the mutations here were limited somewhat based on the crystal structure of Kv1.2 (Long et al., 2005) to those that were predicted to face the inner cavity. Where the literature indicated that an alanine was incompatible with expression, other changes were made as summarized in Table I. The results of this mutational scan of the Kv1.5 deep pore and S6 domains showed a great deal of overlap with key residues shown to be involved in block by other compounds (Caballero et al., 2002; Decher et al., 2004; Decher et al., 2006; Herrera et al., 2005; Yeola et al., 1996). This was not surprising given the limited number of amino acid side chains that project into the inner cavity and are available for interaction with a candidate blocker.

Residues that resulted in a significant change in the vernakalant IC₅₀ included T479, T480, C500, A501, I502, V505, 1508, and P532. Despite the overlap in binding sites, there were apparent differences in both the importance of various residues to block by the various compounds, (compare changes in IC₅₀ for vernakalant and flecamide with mutation at I502, V505 and I508 (FIGS. 5 and 6)), and in the distance along S6 involved (FIG. 9). Block by both AVE0118 and

S0100176 was affected by mutations at L510, V512 and V516 (Decher et al., 2004; Decher et al., 2006). Although L510 and V516 were not screened in the present study, the lack of significant difference in block by vernkalant beyond I508 (FIG. 2), (with particular exception of P532L (see below)), suggests a minor role of those residues in vernakalant's interaction with the channel.

Other Amino Acid Substitutions Gave Further Information on Vernakalant Binding in the S6

In order to gain some additional insight into vernakalant binding to Kv1.5, several further mutations were made at the key residues identified by the alanine scan (FIG. 5 and Table I). The more conservative threonine to serine mutations at 479 and 480, resulted in a similar reduction in side chain volume as alanine but maintained the polar hydroxyl group. Both these mutations resulted in a decrease in potency of the drug (FIG. 5A) and WT-like inactivation rates (not shown). It is difficult to say from these results what exactly the mutations are doing at this site in the base of the selectivity filter. T479 is likely to contribute to K⁺ coordination (Zhou and MacKinnon, 2004) and so mutations here may be having multiple effects on permeation with downstream effects on inactivation. In an oocyte study, a V481 L mutation also made Kv1.5 more sensitive to flecamide without any obvious effects on inactivation (Herrera et al., 2005). This is despite evidence suggesting that this residue is important for K⁺ coordination (Long et al., 2005). As shown in Table I, a significant decrease in potency on mutation of T479 to an alanine but not for the V481 L mutation was observed. T479A has the additional affect of enhancing C-type inactivation (FIG. 2B) which complicates interpretation of the change in IC₅₀. It is interesting to note that this mutation does not have the same effect on drug block by all compounds studied, with some becoming more potent (vernakalant and flecamide, Tables I and II) and others less so (AVE0118, S0100176, and quinidine; (Decher et al., 2004; Decher et al., 2006; Yeola et al., 1996), FIG. 9).

I502, A Critical Residue for Vernakalant Binding

At I502 a phenylalanine was inserted, in addition to the alanine and leucine mutations, to determine the effects of a large aromatic residue at this site. As is shown in FIG. 5A, the IC₅₀ at I502 was inversely related to hydrophobicity and the presence of a residue capable of π-stacking appeared irrelevant. This highlights the potential importance of hydrophobic interactions with I502 in determining inner vestibule architecture. Interestingly, the IC₅₀ of flecamide was increased more by this mutation than by the alanine (FIG. 6), perhaps indicating some steric affects to these mutations. The importance of I502 in flecamide block has been studied previously (Herrera et al., 2005). In that study, the I502L mutation was adequate for rendering Kv1.5 as sensitive to flecamide as Kv3.1 and Kv4.2. Those studies were carried out using Xenopus oocytes and thus the exact IC₅₀ values are different but the trend was the same as observed in the HEK cells used in the present study.

Further evidence that the residue I502 is of critical importance in regulating the potency of vernakalant action on Kv1.5 was obtained from electrophysical studies of the voltage-dependence of block (FIGS. 7, 8). Here it was found that the mutation I502A shifted the calculated binding site of vernakalant within the electric field from 0.17 to 0.39 of the way across the electric field from the inside of the pore. This action of I502A was unique for the sites tested in this manner for vernakalant, and suggests that the mutation is able to specifically disrupt high affinity access of the drug to the permeation pathway, as the IC₅₀ for block was reduced 25-fold in this mutant (FIG. 3). The increased electrical distance that results from the mutation may reflect a deeper penetration of the drug into the inner vestibule after loss of its coordinated site, and an upper limit of intracellular access of the drug due to the proximity of the narrowing of the selectivity filter. If vernakalant is now moving deeper into the pore, the increase in IC₅₀ may also reflect competition with K⁺ for interaction with residues at the base of the filter. In comparison, the similar mutation for flecamide did not change the electrical distance as much (0.32 vs. 0.17), and the potency of block was only altered 2.4-fold (FIG. 4). In flecamide, the V505A mutation was able to induce a similar, if opposite change in potency to I502A, which suggests that flecamide binding to the channel is coordinated along a broader region of S6 than vernakalant and in a less critical manner by the isoleucine at position 502 and the valine at position 505.

Additional mutations at V505 and I508 resulted in generally modest changes in IC₅₀ (FIGS. 5 and 6) and mostly tracked with changes in size, though flecamide in particular may be capable of cation-π-stacking interactions with an aromatic placed at V505 but not at I508. A hydrophobic aromatic more distant from the pore appears more favorable for block by vernakalant, (compare I508F and I508Y in FIG. 5). These mutations are of particular interest because the equivalent residues in the proteins underlying the ventricular delayed rectifier currents I_{Ks} (KvLQT1), and I_{Kr} (hERG), are phenylalanine and tyrosine respectively (FIG. 11). While the I508Y mutant had a similar IC₅₀ to hERG (Fedida et al., 2005), data for I_{Ks} showed that this channel was not sensitive to vernakalant (unpublished data) indicating that the extensive sequence differences between this channel and Kv1.5 (FIG. 11) make any predictions difficult.

One can conceive of several mechanisms by which a given mutation could affect drug block. These include: 1) a loss of a side chain quality needed for direct interaction with the drug (hydrophobic, electrostatic, π-stacking); 2) the mutation leads to local or global changes in binding site without the residue making direct contact with the drug itself, 3) the mutation has a steric effect, blocking access to the binding site; 4) the mutation promotes a change in the gating state of the channel. To illustrate the third point, a naturally occurring polymorphism in Kv1.5, P532L, appears to have just such a steric effect on drug access as was shown for quinidine (Drolet et al., 2005) and propafenone (Simard et al., 2005). Quinidine traversed the same distance across the electric field to block Kv1.5 channels with the P532L mutation, but block was reduced and evidence suggested that in the absence of the proline a helix might form and impede access to the inner vestibule (Drolet et al., 2005). It appears that this mutation may have a similar effect on access for vernakalant (FIG. 2L).

Insights from the Mutational Studies of Kv1.5 Applied to Vernakalant Block of Other Voltage-Gated Ion Channels

An alignment of a range of voltage-gated potassium channels for the S5, selectivity filter and S6 regions is shown in FIG. 11. It can be seen that at the equivalent position of I502, in most of the Kv3 and Kv4 channels, as well as hERG channels, there is a leucine. In the studies described herein, substitution of isoleucine with leucine decreased the sensitivity of Kv1.5 to 31.1 μM, and this is close to the reported sensitivity of Kv4 channels for vernakalant, but somewhat higher than that reported for hERG channels (Fedida et al., 2005). A possible explanation for the difference in the latter case may be the presence of the tyrosine at the equivalent position to I508 in hERG. I508Y increased the IC₅₀ for vernakalant on Kv1.5 to 24.7 μM (Table I), again similar to the reported value for hERG. This tyrosine along with a phenylalanine at the equivalent position of V512 in Kv1.5 (FIG. 11) has been implicated in the binding of several classes of drug to hERG, leading to acquired long QT syndrome, as reviewed in Sanguinetti and Mitcheson, 2005. Thus, the reduced potency for vernakalant on K⁺ channels other than Kv1.5 may reflect, at least in part, the 3-dimensional structural differences inferred from the present study.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Caballero R, Moreno I, Gonzalez T, Valenzuela C, Tamargo J and Delpon E (2002) Putative Binding Sites for Benzocaine on a Human Cardiac Cloned Channel (Kv1.5). Cardiovasc Res 56: pp 104-117.
Choi K L, Mossman C, Aubé J and Yellen G (1993) The Internal Quaternary Ammonium Receptor Site of Shaker Potassium Channels. Neuron 10: pp 533-541.
Decher N, Kumar P, Gonzalez T, Pirard B and Sanguinetti M C (2006) Binding Site of a Novel Kv1.5 Blocker: a “Foot in the Door” Against Atrial Fibrillation. Mol Pharmacol 70: pp 1204-1211.
Decher N, Kumar P, Gonzalez T, Renigunta V and Sanguinetti M C (2005) Structural Basis for Competition Between Drug Binding and Kvbeta 1.3 Accessory Subunit-induced N-Type Inactivation of Kv1.5 Channels. Mol Pharmacol 68: pp 995-1005.
Decher N, Pirard B, Bundis F, Peukert S, Baringhaus K H, Busch A E, Steinmeyer K and Sanguinetti M C (2004) Molecular Basis for Kv1.5 Channel Block: Conservation of Drug Binding Sites Among Voltage-Gated K+ Channels. J Biol Chem 279: pp 394-400.
Drolet B, Simard C, Mizoue L and Roden D M (2005) Human Cardiac Potassium Channel DNA Polymorphism Modulates Access to Drug-Binding Site and Causes Drug Resistance. J Clin Invest 115: pp 2209-2213.
Fedida D (1997) Gating Charge and Ionic Currents Associated With Quinidine Block of Human Kv1.5 Delayed Rectifier Channels. J Physiol 499 (Pt 3): pp 661-675.
Fedida D (2007) Vernakalant (RSD1235): a Novel, Atrial-Selective Antifibrillatory Agent. Expert Opin Investig Drugs 16: pp 519-532.
Fedida D, Orth P M R, Chen J Y C, Lin S, Plouvier B, Jung G, Ezrin A and Beatch G N (2005) The Mechanism of Atrial Antiarrhythmic Action of RSD1235. J Cardiovasc Electrophysiol 16: pp 1227-1238.
Fedida D, Wible B, Wang Z, Fermini B, Faust F, Nattel S and Brown A M (1993) Identity of a Novel Delayed Rectifier Current From Human Heart With a Cloned K+ Channel Current. Circ Res 73: pp 210-216.
Feng J L, Wible B, Li G R, Wang Z G and Nattel S (1997) Antisense Oligodeoxynucleotides Directed Against Kv1.5 mRNA Specifically Inhibit Ultrarapid Delayed Rectifier K+ Current in Cultured Adult Human Atrial Myocytes. Circ Res 80: pp 572-579.
Franqueza L, Longobardo M, Vicente J, Delpón E, Tamkun M M, Tamargo J, Snyders D J and Valenzuela C (1997) Molecular Determinants of Stereoselective Bupivacaine Block of HKv1.5 Channels. Circ Res 81: pp 1053-1064.
Fuster V, Ryden L E, Cannom D S, Crijns H J, Curtis A B, Ellenbogen K A, Halperin J L, Le Heuzey J Y, Kay G N, Lowe J E, Olsson S B, Prystowsky E N, Tamargo J L, Wann S, Smith S C, Jr., Jacobs A K, Adams C D, Anderson J L, Antman E M, Halperin J L, Hunt S A, Nishimura R, Ornato J P, Page R L, Riegel B, Priori S G, Blanc J J, Budaj A, Camm A J, Dean V, Deckers J W, Despres C, Dickstein K, Lekakis J, McGregor K, Metra M, Morais J, Osterspey A, Tamargo J L and Zamorano J L (2006) ACC/AHA/ESC 2006 Guidelines for the Management of Patients With Atrial Fibrillation: a Report of the American College of Cardiology/American Heart Association Task Force on Practice Guidelines and the European Society of Cardiology Committee for Practice Guidelines (Writing Committee to Revise the 2001 Guidelines for the Management of Patients With Atrial Fibrillation): Developed in Collaboration With the European Heart Rhythm Association and the Heart Rhythm Society. Circ 114: pp e257-e354.
Go A S, Hylek E M, Phillips K A, Chang Y, Henault L E, Selby J V and Singer D E (2001) Prevalence of Diagnosed Atrial Fibrillation in Adults: National Implications for Rhythm Management and Stroke Prevention: the AnTicoagulation and Risk Factors in Atrial Fibrillation (ATRIA) Study. JAMA 285: pp 2370-2375.
Grissmer S, Nguyen A N, Aiyar J, Hanson D C, Mather R J, Gutman G A, Karmilowicz M J, Auperin D D and Chandy K G (1994) Pharmacological Characterization of Five Cloned Voltage-Gated K+ Channels, Types Kv1.1, 1.2, 1.3, 1.5, and 3.1, Stably Expressed in Mammalian Cell Lines. Molec Pharmacol 45: pp 1227-1234.
Herrera D, Mamarbachi A, Simoes M, Parent L, Sauve R, Wang Z and Nattel S (2005) A Single Residue in the S6 Transmembrane Domain Governs the Differential Flecamide Sensitivity of Voltage-Gated Potassium Channels. Mol Pharmacol 68: pp 305-316.
Holmgren M, Shin K S and Yellen G (1998) The Activation Gate of a Voltage-Gated K+ Channel Can Be Trapped in the Open State by an Intersubunit Metal Bridge. Neuron 21: pp 617-621.
Holmgren M, Smith P L and Yellen G (1997) Trapping of Organic Blockers by Closing of Voltage-Dependent K+ Channels—Evidence for a Trap Door Mechanism of Activation Gating. J Gen Physiol 109: pp 527-535.
Kannel W B, Wolf P A, Benjamin E J and Levy D (1998) Prevalence, Incidence, Prognosis and Predisposing Conditions for Atrial Fibrillation: Population Based Estimates. Am J Cardiol 82: pp 2N-9N.
Kirsch G E and Drewe J A (1993) Gating-Dependent Mechanism of 4-Aminopyridine Block in Two Related Potassium Channels. J Gen Physiol 102: pp 797-816.
Leung J M, Bellows W H and Schiller N B (2004) Impairment of Left Atrial Function Predicts Post-Operative Atrial Fibrillation After Coronary Artery Bypass Graft Surgery. Eur Heart J 25: pp 1836-1844.
Liu Y and Joho R H (1998) A Side Chain in S6 Influences Both Open-State Stability and Ion Permeation in a Voltage-Gated K+ Channel. Pflugers Arch 435: pp 654-661.
Long S B, Campbell E B and MacKinnon R (2005) Crystal Structure of a Mammalian Voltage-Dependent Shaker Family K+ Channel. Science 309: pp 897-903.
Lopez G A, Jan Y N and Jan L Y (1994) Evidence That the S6 Segment of the Shaker Voltage-Gated K+ Channel Comprises Part of the Pore. Nature 367: pp 179-182.
Persson F, Carlsson L, Duker G and Phd I J (2005) Blocking Characteristics of HKv1.5 and HKv4.3/HKChIP2.2 After Administration of the Novel Antiarrhythmic Compound AZD7009. J Cardiovasc Pharmacol 46: pp 7-17.
Pratt C, Roy D, Juul-Moller S, Torp-Pedersen C, Toft E, Nielson T, Rasmussen S L and et al. (2006) Efficacy and Tolerance of RSD1235 in the Treatment of Atrial Fibrillation or Atrial Flutter: Results of a Phase III, Randomized, Placebo-Controlled, Multicenter Trial. Europace 8: pp 155-3.
Rockson S G and Albers G W (2004) Comparing the Guidelines: Anticoagulation Therapy to Optimize Stroke Prevention in Patients With Atrial Fibrillation. J Am Co/l Cardiol 43: pp 929-935.
Roy D, Pratt C, Wyse D G, Toft E, Torp-Pedersen C, Juul-Moller S and et al. (2005) Efficacy and Safety of RSD1235 in the Treatment of Recent Onset Atrial Fibrillation in ACT I (Atrial Arrhythmia Conversion Trial I), a Phase III, Randomized, Placebo-Controlled, Multi-Center Trial. European Society of Cardiology.
Roy D, Rowe B H, Stiell I G, Coutu B, Ip J H, Phaneuf D, Lee J, Vidaillet H, Dickinson G, Grant S, Ezrin A M and Beatch G N (2004) A Randomized, Controlled Trial of RSD1235, a Novel Anti-Arrhythmic Agent, in the Treatment of Recent Onset Atrial Fibrillation. J Am Coll Cardiol 44: pp 2355-2361.
Simard C, Drolet B, Yang P, Kim R B and Roden D M (2005) Polymorphism Screening in the Cardiac K+ Channel Gene KCNA5. Clin Pharmacol Ther 77: pp 138-144.
Snyders D J, Knoth K M, Roberds S L and Tamkun M M (1992) Time, Voltage-, and State-Dependent Block by Quinidine of a Cloned Human Cardiac Potassium Channel. Mol Pharmacol 41: pp 322-330.
Stiell I G, Roy D, Rowe B H, Pratt C, Dickinson G and Kitt T (2006) Efficacy and Safety of RSD1235 in the Treatment of Acute Atrial Fibrillation. Acad Emerg Med 13, Suppl. 1: pp S162-a.
Woodhull A M (1973) Ionic Blockage of Sodium Channels in Nerve. J Gen Physiol 61: pp 687-708.
Yeola S W, Rich T C, Uebele V N, Tamkun M M and Snyders D J (1996) Molecular Analysis of a Binding Site for Quinidine in a Human Cardiac Delayed Rectifier K+ Channel—Role of S6 in Antiarrhythmic Drug Binding. Circ Res 78: pp 1105-1114.
Zhou Y and MacKinnon R (2004) Ion Binding Affinity in the Cavity of the KcsA Potassium Channel. Biochemistry 43: pp 4978-4982.

1. A method of identifying an ion channel modulating compound, comprising identifying a compound that blocks a Kv1.5 channel comprising Kv1.5 polypeptides, wherein:
(a) said block is reduced when said Kv1.5 polypeptides comprise a mutation in an amino acid residue corresponding to amino acid residue 480, or a region consisting of amino acid residues 500 to 512 of SEQ ID NO:1; and/or
(b) said block is increased when said Kv1.5 polypeptides comprise a mutation in an amino acid residue corresponding to amino acid residue 479 or 501 of SEQ ID NO:1.

2. The method of claim 1, wherein said block is reduced at least 5-fold when said Kv1.5 polypeptides comprise a mutation in an amino acid residue corresponding to amino acid residue 502 of SEQ ID NO:1.

3. The method of claim 1, wherein said block is reduced when said Kv1.5 polypeptides comprise a mutation in an amino acid residue corresponding to an amino acid residue selected from 480, 500, 502, 505, and 508 of SEQ ID NO:1.

4. The method of claim 3, wherein said amino acid residue corresponds to amino acid residue 505 or 508.

5. The method of claim 1, wherein said block is not significantly reduced when said Kv1.5 polypeptides comprise a mutation in a region corresponding to amino acid residues 509-531 of SEQ ID NO:1.

6. The method of claim 5, wherein said Kv1.5 polypeptides comprise a mutation in an amino acid residue corresponding to an amino acid residue selected from 510, 512, and 516 of SEQ ID NO:1.

7. The method of claim 1, wherein said ion channel modulating compound is an atrial-selective antiarrhythmic agent.

8. The method of claim 1, wherein said ion channel modulating compound acts preferentially on K⁺ channels as compared to Na⁺ channels.

9. The method of claim 1, wherein said block is determined in cells expressing an exogenous wild type or mutant Kv1.5 polypeptide.

10. The method of claim 9, wherein said cells comprise an exogenous polynucleotide that encodes a wild type or mutant Kv1.5 polypeptide.

11. The method of claim 10, wherein said polynucleotide was introduced into said cells by transient transfection.

12. The method of claim 1, wherein said block is determined by whole cell current recordings.

13. An ion channel modulating compound identified according to a method of any one of claims 1-12.
